(19) United States
(12) Patent Application Publication
(10) Pub. No.: US 2023/0000921 A1
(43) Pub. Date: Jan. 5, 2023

(54) CHANGING GUT MICROBIOTA TO REDUCE ALCOHOL CRAVING

(71) Applicant: VIRGINIA COMMONWEALTH UNIVERSITY, Richmond, VA (US)

(72) Inventor: Jasmohan BAJAJ, Richmond, VA (US)

(21) Appl. No.: 17/780,728

(22) PCT Filed: Nov. 2, 2020
(86) PCT No.: PCT/US2020/058466

§ 371 Date: May 27, 2022

Related U.S. Application Data
(60) Provisional application No. 62/932,618, filed on Nov. 8, 2019.

(51) Int. Cl. A61K 35/24; A61K 35/741; A61P 25/32

(52) U.S. Cl. CPC A61K 35/24; A61K 35/741; A61P 25/32

(57) ABSTRACT

A composition of fecal material is prepared and administered to a subject suffering from alcohol craving, alcohol consumption and/or alcohol use disorder. The donor fecal material is enriched in beneficial microbiota associated with reduced alcohol craving and/or consumption and supplies beneficial microbiota in which the recipient is deficient, and reduces harmful microbes. Methods of preparing and administering are disclosed, along with the identification of beneficial and harmful microbiota families. The treatment is also useful for delivery of microbiota that are short chain fatty acid producers to reduce alcohol craving and/or consumption in subjects deficient in short chain fatty acids.

### CROSS-REFERENCE TO RELATED APPLICATIONS

This application claims benefit of U.S. Provisional Application No. 62/932,618, filed Nov. 8, 2019, the complete contents of which is hereby incorporated by reference.

### STATEMENT OF FEDERALLY SPONSORED RESEARCH AND DEVELOPMENT

This invention was made with government support from United States Department of Veterans Affairs Merit review 210CX00176 and National Institutes of Health National Center for Advancing Translational Sciences R21TR002024. The United States government has certain rights in the invention.

### FIELD OF THE INVENTION

The invention generally relates to treatments for alcohol use disorder associated with microbial alterations that worsen with cirrhosis. The invention further relates to methods of fecal microbiota transplantation as a treatment for alcohol use disorder.

### BACKGROUND

Alcohol use disorder (AUD) and alcohol-related cirrhosis are major causes of morbidity and mortality. Continued alcohol misuse in the setting of end-stage organ damage such as cirrhosis can hasten disease progression. Therefore, therapies that encourage alcohol abstinence or reduce alcohol misuse are relevant. Pharmacotherapies for craving in alcohol-related cirrhosis have yielded partial success and largely focus on neuromodulation. AUD is associated with major changes in the gut-brain axis that is worsened with the occurrence of cirrhosis. In addition, other studies have demonstrated a potential role of microbiota in the addictive behavior. For example, see Cryan et al. (Physiol Rev 2019; 99:1877-2013) and Liang et al. (Front Integr Neurosci 2018; 12:33). These and other studies have demonstrated the potential effects of the microbiota on the gut-brain axis and neuronal circuits that regulate behaviors, including addictive behavior.

In patients with advanced cirrhosis who are not currently drinking alcohol, the altered gut-brain axis responds to fecal microbiota transplant (FMT) (6-8). For example, see publications by Bajaj et al. in (Hepatology 2017; 66:1727-1738), Gastroenterology 2019 (May; 156(6):1921-1923), Hepatology 2018 (Hepatology. 2018 October; 68(4):1549-1558) (JCI Insight 2019 JCI Insight. 2019 Dec. 19; 4(24):e133410), and (Hepatology 2019 Hepatology. 2019 November; 70(5):1690-1703), which demonstrated safety and efficacy of FMT as a treatment for hepatic encephalopathy and the ability to effect changes in the microbiota of FMT recipients.

Thus, abstinence from alcohol is linked with microbial change. However, the impact of microbial modulation to facilitate reduction of alcohol misuse remains unclear. There is a need to identify a useful application of microbial modification that would have a meaningful impact on alcohol cravings underlying the morbidity and mortality associated with AUD, including alcoholism and alcohol misuse or abuse. It would further be beneficial to have a treatment that would modify the microbiota and reduce alcohol craving, thereby encouraging abstinence and reducing alcohol misuse.

### SUMMARY OF THE INVENTION

An aspect of the invention pertains to the use of fecal material and its transfer to a recipient as a treatment for AUD that modulates cognition, behavior, daily function, short-term alcohol craving and alcohol consumption by changing the microbial composition and function in the FMT recipient.

In one embodiment, the invention is a method of reducing alcohol craving in a subject in need thereof, comprising the steps of assaying a stool sample from the subject to identify microbiota species resident in the subject's colon and determine whether the subject is deficient in beneficial microbiota species, obtaining a sample of fecal material from a suitable donor wherein the sample of fecal material comprises beneficial microbiota, which are associated with a reduction in alcohol craving and/or consumption, processing the sample of fecal material to produce at least one dose for fecal material transfer (FMT), and administering a dose of FMT to the subject.

As used herein, the term “beneficial microbiota” refers to species associated herein with reduced alcohol craving, alcohol misuse and/or alcohol use disorder. It is an object of the invention to provide beneficial microbiota to the subject. The microbiota identified herein as being beneficial include species, genera and strains belonging to Lachnospiraceae and Ruminococcaceae families such as, Odoribacter, Blautia, Roseburia, in addition to other taxa such as Alistipes, Eubacterium, Lactonifactor, Oscillibacter, Anaerostipes and Bilophila. Thus, at minimum, the microbiota in the sample of fecal material comprises at least one species selected from the group consisting of strains belonging to Lachnospiraceae, Ruminococcaceae, Odoribacter, Blautia, Alistipes and Bilophila.

In one embodiment of the invention, the method includes an administering step to deliver a dose of FMT. In one embodiment, the adminstering is performed as an enema comprising the dose of FMT. In another embodiment, the subject receives multiple doses that are combined into a single administration by enema. When administered as an enema, the enema is retained in the colon for at least thirty minutes. In another embodiment, administration is in the form of a rectal suppository. In yet another embodiment, the FMT is lyophilized and packaged in capsules having an enteric coating and the capsules are administered orally. In another embodiment more than one dose of FMT is administered as a series over a suitable period of time.

In one embodiment of the invention, the method comprises a step of screening for pathogens selected from the group consisting of Clostridium difficile toxin B, qualitative RT-PCR, Cyclospora and Isospora examination, ova and parasites exam with Giardia antigen EIA, Salmonella-Shigella-Campylobacter culture, Shiga toxins EIA with reflex to E. coli O157 culture and Vibrio culture, Cryptosporidium antigen EIA, Helicobacter pylori antigen EIA, stool norovirus EIA, stool rotavirus antigen detection, adenovirus antigen detection, gastroenteritis EIA, vancomycin-resistant Enterococcus culture and Microsporidia exam.

In one embodiment of the invention, the method further comprises the steps of filtering the sample of fecal material, suspending the filtered fecal material in a pharmaceutically acceptable buffered solution to produce doses of FMT, storing the doses of FMT at −80° C. until time of administration. The doses are thawed when a practitioner and subject are ready for the administering step.

In another embodiment of the invention, the method further comprises the steps of obtaining a second stool sample from the subject at least 15 days after the administering step, culturing the second stool sample to identify microbiota species resident in the subject's colon after receiving the at least one dose of FMT, and determining whether the microbiota of the subject has been altered by receiving the at least one dose of FMT. If the desired results have not been achieved, another dose of FMT is administered at least one time. Multiple doses may be administered, and these would typically be performed at 15-day intervals.

In yet another embodiment, the invention is method of increasing or changing the composition of short-chain fatty acids (SCFA) in the colon of a subject in need thereof, comprising the steps of assaying a stool sample from the subject to identify microbiota species resident in the subject's colon, obtaining a sample of fecal material from a suitable donor wherein the sample of fecal material comprises microbiota known to alter short-chain fatty acids associated with a reduction in alcohol craving and/or consumption, processing the sample of fecal material to produce at least one dose for fecal material transfer (FMT), and administering a dose of FMT rectally to the subject. The administering step may be performed as an enema comprising the dose of FMT. When administered as an enema, the enema is retained in the colon for at least thirty minutes. In another embodiment, the subject receives more than one dose of FMT, or multiple doses that are combined into a single administration by enema. In another embodiment, administration is performed as a rectal suppository. In yet another embodiment, FMT is lyophilized and packaged in capsules having an enteric coating and the capsules are administered orally. The beneficial microbiota in the dose of fecal material comprise at least one species selected from the group consisting of Lachnospiraceae, Ruminococcaceae, Odoribacter, Blautia, Alistipes, Roseburia, Eubacterium, Lactonifactor, Oscillibacter, Anaerostipes and Bilophila. In one embodiment, the microbiota in the sample of fecal material comprises at least one species selected from the group consisting of strains belonging to Lachnospiraceae, Ruminococcaceae, Odoribacter, Blautia, Alistipes and Bilophila. Confirmation of a therapeutically effective treatment can be made by measuring SCFA in a stool sample or in plasma at a suitable time after the treatment is administered.

In one embodiment of the invention, the method further comprises the steps of obtaining a second stool sample from the subject at least 15 days after the administering step, culturing the second stool sample to identify microbiota species resident in the subject's colon after receiving the at least one dose of FMT, and determining whether the microbiota of the subject has been altered by receiving the dose of FMT. In some cases, after at least 15 days, another dose of FMT is administered at least one time. In another embodiment of the invention, the patient is routinely administered additional doses of FMT at suitable intervals.

In practicing the method of the invention, the step of processing may comprise screening the sample of fecal material for pathogens, filtering the sample of fecal material, suspending the filtered fecal material in a pharmaceutically acceptable buffered solution to produce the at least one dose of FMT, storing the at least one dose of FMT at −80° C. until time of administration, and thawing the at least one dose of FMT for the administering step.

The step of screening the sample of fecal material for the pathogens may include any or all selected from the group consisting of Clostridium difficile toxin B, qualitative RT-PCR, Cyclospora and Isospora examination, ova and parasites exam with Giardia antigen EIA, Salmonella-Shigella-Campylobacter culture, Shiga toxins EIA with reflex to E. coli O157 culture and Vibrio culture, Cryptosporidium antigen EIA, Helicobacter pylori antigen EIA, stool norovirus EIA, stool rotavirus antigen detection, adenovirus antigen detection, gastroenteritis EIA, vancomycin-resistant Enterococcus culture and Microsporidia exam.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIGS. 1A-1F show scoring for FMT and placebo groups pre- and post-treatments. FIGS. 1A and 1B show alcohol craving for FMT and placebo groups, respectively. FIGS. 1C and 1D show total SIP score for FMT and placebo groups, respectively. FIGS. 1E and 1F show psychological SIP score for FMT and placebo groups, respectively.

FIG. 2 shows a schematic diagram of the experimental design for a study.

FIG. 3 shows a CONSORT flow diagram of participation in the study.

FIGS. 4A-4C show changes in ACQ in study subjects. FIG. 4A shows pre- to post-treatment reduction (improvement) in ACQ in FMT, but not in placebo compared to baseline (4B) and changes in the delta from baselines (4C).

FIGS. 5A-5C show psychosocial sickness impact profile (SIP) in study subjects. SIP is reduced (improved) in FMT (5A) but not in placebo compared to their baseline (5B) and changes in the delta from baselines (5C).

FIGS. 6A-6D show measurements of serum IL-6 and lipopolysaccharide-binding protein (LBP) in study subjects. FIGS. 6A and 6B show that serum IL-6 is lowered in the FMT-treated group, but not in the placebo-treated group, respectively, compared to their starting pre-treatment baseline. FIGS. 6C and 6D show a reduction in serum LBP in the FMT-treated group but not in the placebo group, respectively, compared to their pre-treatment baselines.

FIGS. 7A-7C show changes in microbiota composition. FIG. 7A shows an increase in Shannon diversity in FMT-treated subjects, but not in (7B) placebo-treated subjects. Change in Shannon diversity was significant between FMT versus placebo, as shown in FIG. 7C.

FIGS. 8A and 8B show linear discriminant analysis effect size (LeFSe). FIG. 8A shows that the LeFSe was higher for Odoribacter and (8B) Bilophila after FMT-treatment compared to baseline. The X axis has the log10 linear discriminant analysis score. FIG. 8B shows that the LeFSe was higher for Alistipes and Roseburia post-FMT treatment compared to post-placebo treatment; the X axis has the log 10 linear discriminant analysis score.

FIG. 9 shows the post-placebo correlation network differences centered around ACQ. Ovals are bacterial genera while the diamond is ACQ. A high score on ACQ indicates higher craving. Bacteria marked with * positive in post-placebo but absent post-FMT, Bacteria marked with † negative in post-placebo but absent post-FMT, Bacteria marked with # negative in post-FMT but absent post-placebo; and Bacteria marked with ** positive post-placebo and negative post-FMT. Potentially beneficial genera such as Eubacterium, Lactonifactor and Ruminococcus were associated with low ACQ after FMT but not after placebo. The reverse pattern was seen with potential pathobionts such as Salmonella, Serratia and Pseudomonas. Ethanoligenens, which is associated with endogenous alcohol production, was negatively linked with ACQ in post-FMT but positive in post-placebo patients.

FIGS. 10A-10C show various pre-FMT vs. post-FMT correlation network differences in microbial genera (ovals) centered around SCFA (hexagons). † marked bacteria that have a negative linkage post-FMT but pre-FMT. Remaining bacteria had positive linkages with their neighboring connecting nodes post-FMT but not before FMT. Prevotella, Ruminococcus, Sutterella and Fusicatenibacter (10A) had positive interactions with each other after-FMT but not pre-FMT. Alistipes, Oscillibacter (10B) and Eryspelotrichaceae (10C) family members were positively linked with plasma SCFA (2-methylbutyric acid and Propionic acid) in post-FMT but not pre-FMT. Escherichia/Shigella was negative in post-FMT but not pre-FMT in linkage with beneficial Alistipes. Positive linkages were found between Ruminococcaceae and Lachnospiraceae constituents and SCFA in plasma and stool post-FMT but not pre-FMT.

FIG. 11 shows the post-FMT correlation network differences in microbial genera (ovals) centered around ACQ (diamond). *marked bacteria that have a negative linkage with ACQ pre-FMT but did not have a linkage with ACQ after FMT. Rest of the bacteria had a negative linkage with ACQ but did not have any correlation before FMT. Beneficial genera and those higher in post-FMT, including Bilophilia, Ruminococcus, Parabacteroides and Succinispira were associated with low ACQ after FMT and were increased over the levels measured at baseline. The reverse pattern was seen with potential pathobionts such as Salmonella. Salmonella was negative pre-FMT and remained absent post-FMT.

FIGS. 12A and 12B show post-placebo vs post-FMT correlation network differences of microbiota (ovals) centered around SCFA (hexagons), including isovaleric acid, butyric acid, propionic acid and acetic acid in plasma, and butyric acid, hexonic acid and acetic acid in stool. *marked bacteria that have a negative linkage with their connected neighboring node in Post-FMT but not post-placebo patients. All other linkages were positive in Post-FMT but not post-placebo. Clostridium, Anaerostipes, Roseburia, Butyricicoccus, Lachnospiracea and Odoribacter were positively linked with each other and with plasma SCFAs (isovaleric acid, propionic acid, acetic acid and butyric acid) in post-FMT, shown in FIG. 12A, but not post-placebo, shown in FIG. 12B; Lactobacillus and Ethanoligenens (marked with *) were negatively linked with stool and plasma SCFA (butyric acid and hexanoic acid) in post-FMT but not post-placebo. Those positively linked included Oscillibacter and Alistipes, which were higher post-FMT with plasma propionate. Erysipelothricaceae was associated with 2-methylbutyric acid post-FMT.

FIGS. 13A and 13B show a comparison of serious adverse events over 6 months. Serious adverse events were defined as hospitalizations or ER visits within 6 months of the intervention. AUD-related events were adjudged based on review of the medical record, and patient interview. FIG. 13A shows the number of placebo- and FMT-treated subjects having SAEs. There was a higher proportion of patients with AUD-related and other SAEs in FMT-treated patients versus placebo-treated patients. FIG. 13B shows median and 95% CI and individual numbers of SAEs per subject over 6 months. The per subject SAE rate was higher in the placebo-treated group.

### DETAILED DESCRIPTION

The following descriptions and examples illustrate some exemplary embodiments of the disclosed invention in detail. Those of the skill in the art will recognize that there are numerous variations and modifications of this invention that are encompassed by its scope. Accordingly, the description of a certain exemplary embodiment should not be deemed to limit the scope of the present invention.

The invention is a composition comprising fecal material and method for its transfer to a recipient as a treatment for AUD that modulates cognition, behavior, daily function, short-term alcohol craving and alcohol consumption by changing the microbial composition and function in the FMT recipient.

As used herein, the following acronyms refer to the indicated terms: AUD: alcohol use disorder; HE: hepatic encephalopathy; FMT: fecal microbiota transplant; SCFA: short-chain fatty acids; AUDIT: alcohol use disorder identification test; ACQ-SF: alcohol craving questionnaire-short form; PHES: psychometric hepatic encephalopathy score; SIP: sickness impact profile; QOL: quality of life; LEfSe: linear discriminant analysis effect size; Etg: ethylglucuronide; LBP: lipopolysaccharide binding protein and SAE: serious adverse event.

The invention is a composition comprising fecal material and method for its transfer to a recipient as a treatment for AUD that modulates cognition, behavior, daily function, short-term alcohol craving and alcohol consumption by changing the microbial composition and function in the FMT recipient. It is an object of the invention to provide beneficial microbiota to the subject.

As used herein, the term “microbiota” refers to the microorganisms that reside in the colon of mammals, particularly in humans. The microbiota are an ecological community of commensal, symbiotic microorganisms, also known as gut flora or gut microbiota. These include bacteria, archaea and fungi that live in the digestive tract, and can also include pathogens. Microbiota of the gut are collectively known as the “microbiome” of the gut.

As used herein, the term “beneficial microbiota” refers to species associated herein with reduced alcohol craving, alcohol misuse and/or alcohol use disorder. The microbiota identified herein as being beneficial include Lachnospiraceae, Ruminococcaceae, Odoribacter, Blautia, Alistipes, Roseburia, Eubacterium, Lactonifactor, Oscillibacter, Anaerostipes and Bilophila. At minimum, the microbiota in the sample of fecal material comprises at least one species selected from the group consisting of Lachnospiraceae, Ruminococcaceae, Odoribacter, Blautia, Alistipes and Bilophila.

As used herein, the phrase “gut-brain axis” refers to a bidirectional communication between the central and the enteric nervous system, linking emotional and cognitive centers of the brain with peripheral intestinal functions. Recent advances in research have described the importance of gut microbiota in influencing these interactions. This interaction between microbiota and GBA appears to be bidirectional, namely through signaling from gut-microbiota to brain and from brain to gut-microbiota by means of neural, endocrine, immune, and humoral links.

In one embodiment, the invention is a method of reducing alcohol craving in a subject in need thereof. As used herein, the terms “reducing alcohol craving”, “reducing alcohol consumption” are used interchangeably, since a reduction in alcohol craving frequently allows a subject afflicted with AUD to reduce their alcohol consumption.

The gut microbiota may be evaluated either by assessing its composition or by measuring its functions. It is an object of the invention to provide SCFA producing microbiota to individuals who are deficient in SCFA. In one embodiment, the invention is a method of assessing microbial metabolites that are SCFA as an indicator of microbiota functions. SCFA are products of fermentation of unabsorbed food residues (mainly carbohydrates) within the colon and are absorbed and used as an energy source for the host or FMT recipient. SCFA also play an important role in the communication between the gut microbiota and other parts of the body. The production of SCFAs is dependent on the microbiota; thus, assessment of SCFAs is a recognized measure of gut microbiota function. In another embodiment, the invention is a method of increasing SCFA production by microbiota. Increased levels of SCFA in stool and/or plasma are associated with reduced alcohol craving and/or consumption. A subject's SCFA levels may be measured before or after treatment in stool or plasma samples since the increased SCFA production occurs in the colon and, as a result of absorption, SCFA are taken up into the blood.

The methods of the invention generally includes assaying a stool sample from the subject to identify microbiota species resident in the subject's colon, obtaining a donor sample of fecal material comprising microbiota associated with a reduction in alcohol craving and/or consumption, processing the sample of fecal material to produce at least one dose for fecal material transfer (FMT), and administering a dose of FMT rectally to the subject. It is an object of the invention to identify a donor able to supply fecal material that is enriched in beneficial microbiota. The ideal donor will have fecal material that is high in as many beneficial microbiota as possible, including any of Lachnospiraceae, Ruminococcaceae, Odoribacter, Blautia, Alistipes, Roseburia, Eubacterium, Lactonifactor, Oscillibacter, Anaerostipes and Bilophila species. Accordingly, the ideal donor will have fecal material that is low in pathobionts, including Ethanoligenens, Streptococcus, Salmonella, Pseudomonas, Enterococcus, and/or Serratia species. These pathobionts are also known to be harmful to the intestinal barrier and can also produce endogenous alcohol, which is counterproductive to reducing alcohol craving and/or consumption.

In one embodiment, the fecal material acquired from a donor may be “spiked” or supplemented with cultures of one or more of beneficial microbiota that are deficient or absent from the donor FMT. In another embodiment, the fecal material of two or more donors may be combined in order to enhance the microbiota content of the final FMT product for administration to a recipient.

In one embodiment, the administering step is performed as an enema comprising the dose of FMT. In another embodiment, the subject receives more than one dose of FMT, or multiple dose that are combined into a single administration by enema. In another embodiment, administration is performed as a rectal suppository. In yet another embodiment, FMT is lyophilized and packaged in capsules having an enteric coating and the capsules are administered orally. When administered as an enema, the enema is retained in the colon for at least thirty minutes.

In one embodiment of the invention, the method comprises the step of processing further comprises screening for pathogens selected from the group consisting of Clostridium difficile toxin B qualitative RT-PCR, Cyclospora and Isospora examination, ova and parasites exam with Giardia antigen EIA, Salmonella-Shigella-Campylobacter culture, Shiga toxins EIA with reflex to E. coli O157 culture and Vibrio culture, Cryptosporidium antigen EIA, Helicobacter pylori antigen EIA, stool norovirus EIA, stool rotavirus antigen detection, adenovirus antigen detection, gastroenteritis EIA, vancomycin-resistant Enterococcus culture and Microsporidia exam.

In one embodiment of the invention, the method further comprises the steps of filtering the sample of fecal material, suspending the filtered fecal material in a pharmaceutically acceptable buffered solution to produce doses of FMT, storing the doses of FMT at −80° C. until time of administration, and thawing them when ready for the administering step.

In another embodiment of the invention, the method further comprises the steps of obtaining a second stool sample from the subject at least 15 days after the administering step, culturing the second stool sample to identify microbiota species resident in the subject's colon after receiving the at least one dose of FMT, and determining whether the microbiota of the subject has been altered by receiving the at least one dose of FMT. If the desired results have not been achieved, another dose of FMT is administered at least one time. Multiple doses may be administered, and these would typically be performed at 15-day intervals. These intervals allow time for the transplanted fecal microbiota to expand in the colon of the subject or patient who is the recipient or host of the FMT. Thus, in one embodiment of the invention, a post-FMT stool sample is collected and assayed to identify whether beneficial microbiota species have increased. An increase in beneficial species is a successful FMT, while a result of no change or a decrease in beneficial species is an unsuccessful FMT.

In addition to increasing beneficial microbiota, FMT reduces microbes that are harmful to the intestinal barrier and could also produce endogenous alcohol. These include Ethanoligenens, Streptococcus. Salmonella, Pseudomonas, Enterococcus, and Serratia species. Thus, in one embodiment, a stool sample is collected from a recipient subject post-FMT, and the sample is assayed to identify whether any of Ethanoligenens, Streptococcus, Salmonella, Pseudomonas, Enterococcus, and/or Serratia species are decreased, indicating a successful FMT. An increase or no change in harmful species would indicate an unsuccessful FMT.

In yet another embodiment, the invention is method of changing short-chain fatty acids (SCFA) in the colon of a subject in need thereof. SCFA of interest include but are not limited to butyric acid, isobutyric acid, acetic acid, propionic acid, valeric acid, isovaleric acid, 2-methybutyric acid, and hexanoic acid. In one embodiment, the method comprises the steps of assaying a stool sample from the subject to identify microbiota species resident in the subject's colon to determine whether the subject is deficient in beneficial microbiota species, obtaining a sample of fecal material from a suitable donor wherein the sample of fecal material comprises microbiota known to alter short-chain fatty acids associated with a reduction in alcohol craving and/or consumption, processing the sample of fecal material to produce at least one dose for fecal material transfer (FMT) and administering a dose of FMT rectally to the subject. The administering step is performed as an enema comprising the dose of FMT. When administered as an enema, the enema is retained in the colon for at least thirty minutes. In another embodiment, the subject receives more than one dose of FMT, or multiple doses that are combined into a single administration by enema. In another embodiment, administration is performed as a rectal suppository. In yet another embodiment, FMT is lyophilized and packaged in capsules having an enteric coating and the capsules are administered orally. The beneficial microbiota in the dose of fecal material comprise at least one species selected from the group consisting of Lachnospiraceae, Ruminococcaceae, Odoribacter, Blautia, Alistipes and Bilophila.

In one embodiment of the invention, the method further comprises the steps of obtaining a second stool sample from the subject at least 15 days after the administering step, culturing the second stool sample to identify microbiota species resident in the subject's colon after receiving the at least one dose of FMT, and determining whether the microbiota of the subject has been altered by receiving the dose of FMT. In one embodiment of the invention, the microbiota is considered to be beneficially altered when any of Lachnospiraceae, Ruminococcaceae, Odoribacter, Blautia, Alistipes, Roseburia, Eubacterium, Lactonifactor, Oscillibacter, Anaerostipes and/or Bilophila species are increased in the post-FMT stool sample. In another embodiment, the microbiota is further considered to be beneficially altered when any of Ethanoligenens, Streptococcus, Salmonella, Pseudomonas, Enterococcus, and/or Serratia species are decreased in the post-FMT stool sample. In another embodiment, the levels of SCFA are measured in plasma. In some cases, after at least 15 days, another dose of FMT is administered at least one time. In another embodiment of the invention, the patient is routinely administered additional doses of FMT at suitable intervals. It is well-known that alcohol craving is an ongoing condition or symptom of AUD, and in some instances, additional doses of FMT may be administered routinely for an indefinite period of time, including many years of repeated dosing.

In practicing the method of the invention, the steps of processing may comprise screening the sample of fecal material for pathogens, combining samples from two or more donors, filtering the sample of fecal material, suspending the filtered fecal material in a pharmaceutically acceptable buffered solution to produce a dose of FMT. The fecal material may be homogenized in a buffered solution and filtered, or it may be pressed through a filter and suspended in the buffered solution. The buffered solution is generally an aqueous solution but may be any pharmaceutically acceptable solvent that is suitable for maintaining the microbiota of the fecal sample. Examples include buffer systems prepared by dissolving an organic acid, e.g. citric and acetic acid, or an inorganic acid, e.g. phosphoric acid in water or preferably in a pharmaceutically acceptable vehicle and adjusting the pH with a base, preferably alkali base, e.g. KOH and NaOH, most preferably NaOH. Alternatively a buffer system can be prepared by dissolving an organic acid, e.g. citric and acetic acid, or inorganic acid, e.g. phosphoric acid, with the appropriate conjugate base, e.g. trisodium citrate, sodium dihydrogen phosphate or sodium acetate, in water or preferably in a pharmaceutically acceptable intravenous vehicle. Optionally the pH can be (fine) adjusted to the final desired pH with hydrochloric acid or sodium hydroxide. Optionally in addition to the tonicity agents which are chosen from glucose, sodium chloride, glycerol, sorbitol, mannitol, fructose, dextran 40 and dextran 70, the buffered formulations of the pharmaceutical compositions can contain a pharmaceutically acceptable adjuvant. This adjuvant is chosen from co-solvents, stabilizers, cryoprotective agents, desiccants, fillers. The fillers and cryoprotective agents are preferably chosen from simple sugars, for example, glucose, mannitol, fructose or sorbitol, disaccharides, for example, sucrose, lactose, trehalose or maltose; or water-soluble polymers, for example dextran, carboxymethylcellulose, polyvinylpyrrolidone or gelatin.

It is important to understand that when someone with AUD without advanced liver disease, such as cirrhosis, stops drinking, the abundance of some harmful bacteria in the microbiome may be reduced in their system. For example, see Gao et al, Front Physiol 2020; 11:370; and also see Leclercq et al. Proc Natl Acad Sci USA 2014; 111:E4485-4493, who showed that when such individuals voluntarily stopped drinking, these changes generally follow the expected pattern of reduction of harmful microbiota. However, some people, especially in the study by Leclercq et al., did not show changes in their microbiota function after stopping problem drinking. Despite this, the use of microbiota as a therapy to reduce alcohol craving or consumption was never demonstrated or suggested by Leclerq et al. or Gao et al. Furthermore, these patients did not have advanced liver disease and were not identified as having tried and failed at other therapies. The invention is a totally novel way of modulating the gut-brain axis in humans, while Leclercq and Gao merely followed what happens when people change drinking habits under supervision.

Before exemplary embodiments of the present invention are described in greater detail, it is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

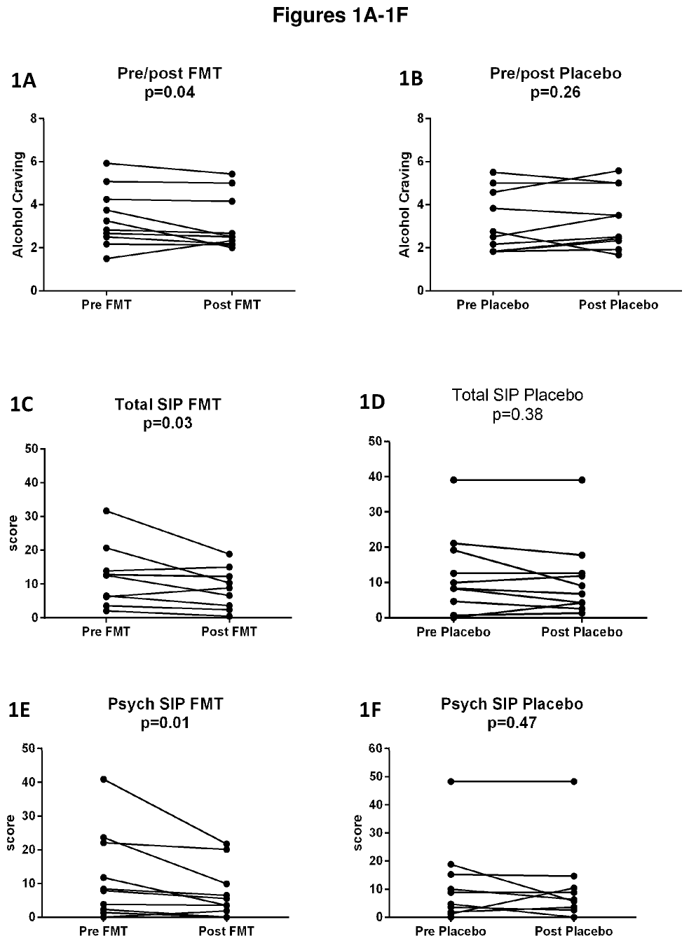

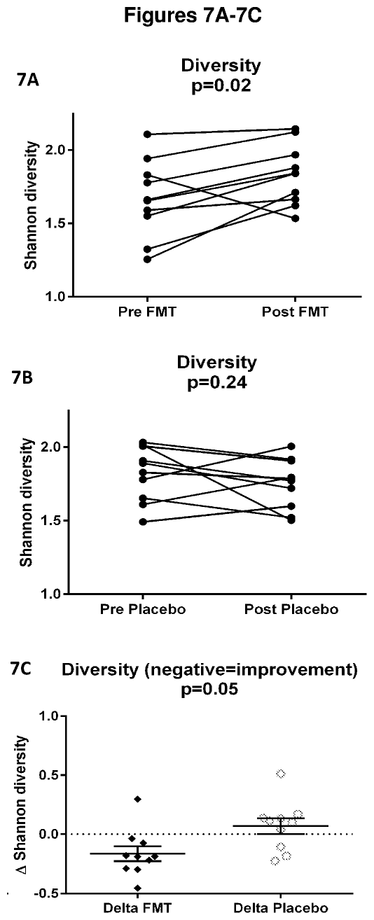

The following Examples provide exemplary compositions and treatments of the invention. These Examples describe materials and methods for using embodiments illustrated in FIGS. 1-13. Additional details can be found in the section entitled “Brief Description of the Drawings”.

In the following examples, AUD-related cirrhosis patients diagnosed with problem drinking (diagnosed as having a score of AUDIT-10>8) were randomized 1:1 into two groups. A control group received at least one placebo enema and a test group received at least one FMT enema from a donor enriched in Lachnospiraceae and Ruminococcaceae as part of a double-blind, randomized clinical trial and demonstrate safety, conducted over a 6-month period. The Examples demonstrate that FMT is safe and associated with short-term reduction in alcohol craving and consumption with favorable microbial changes versus placebo in alcohol-related cirrhosis who were active alcohol misusers. There was also a reduction in AUD-related events over 6 months in patients assigned to FMT.

### Example 1

### Overview

Alcohol craving questionnaire, alcohol consumption (urinary ethylglucuronide/creatinine, Etg), quality of life (QOL), cognition, serum IL-6 and lipopolysaccharide-binding protein (LBP), plasma/stool short-chain fatty acids (SCFA) and stool microbiota were tested at baseline and day 15. A 6-month follow-up with serious adverse events (SAE) analysis was performed. 20 patients with AUD-related cirrhosis [65±6.4 years, all men, MELD 8.9±2.7] with similar demographics, cirrhosis and AUD severity were included. Craving reduced significantly in 90% of FMT versus 30% in placebo at day 15 (p=0.02) with lower urinary Etg (p=0.03), improved cognition and psychosocial QOL. There was reduction in serum IL-6 and LBP and increased butyrate/isobutyrate compared to baseline in FMT but not placebo. Microbial diversity increased post-FMT with higher Ruminococcaceae and other beneficial taxa but was not increased in subjects receiving placebo. Particular microbial species were linked with increased SCFA levels, and diversity of these SCFA-producing taxa were also increased. At 6 months, patients with any SAEs (8 vs 2, p=0.02), AUD-related SAEs (7 vs 1, p=0.02) and SAEs/patient [median(IQR),1.5(1.25) vs 0(0.25) in FMT,p=0.02] were higher in placebo versus FMT.

### FMT Donor Selection

To select a donor of fecal material, 16S rRNA sequencing data was obtained from active drinkers with cirrhosis and healthy control samples (n=156) from Virginia Commonwealth University and McGuire VA Medical Center to train a Random Forest Classifier, a machine learning classification technique. The resulting classifier (AUC=0.94) was used to classify OpenBiome stool donors, giving each donor a classification score that classifies the sample as ‘healthy’. In addition, the relative abundances of Lachnospiraceae and Ruminococcaceae bacterial families in each donor were calculated, and taxa found to be depleted in those with alcohol intake patients were identified. These data were combined to rank OpenBiome stool donors (n=28), from which the donor with the highest aggregate ranking was selected as the FMT donor for FMT-randomized patients. The selected donor had the highest relative abundances of Lachnospiraceae, and Ruminococcaceae among the stool donor registry. The stool sample from this donor was aliquoted and used for all recipients in the FMT-assigned groups.

### OpenBiome Stool Donor Preparation

FMT was prepared from the healthy volunteer screened by OpenBiome and selected as described in the previous step and also in keeping with FMT best practices (see Addolorate et al. J Hepatol 2016:65:618-630). Briefly, qualified donors must pass a 178-point clinical assessment for infectious and microbiome-mediated diseases and 30 stool pathogen and serological tests before and after the stool is collected. These tests include:

in stool: Clostridium difficile toxin B qualitative RT-PCR, Cyclospora and Isospora examination, ova and parasites exam with Giardia antigen EIA, Salmonella-Shigella-Campylobacter culture, Shiga toxins EIA with reflex to E. coli O157 culture and Vibrio culture, Cryptosporidium antigen EIA, Helicobacter pylori antigen EIA, stool norovirus EIA, stool rotavirus antigen detection, adenovirus antigen detection, gastroenteritis EIA, vancomycin-resistant Enterococcus culture and Microsporidia exam;

in serum: human immunodeficiency virus ½ antigen and Ab, 4^{th} generation with reflexes, hepatitis A IgM Ab, hepatitis B core Ab, total; hepatitis B Core Ab IgM; hepatitis B surface antigen with reflex, hepatitis C Ab, Treponema pallidum (syphilis) Ab cascading reflex with RPR reflex to titer, hepatic function panel, HTLV-I/II Ab, with reflex to confirmatory assay; and

in whole blood: complete blood count (CBC), including differential and platelets. This testing regimen has been recently published in Bajaj Nat Rev Gastroenterol Hepatol 2019; 16:235-246.

Donor stool is collected within 6 hours of passage. Stool (12.5 g) is combined with a glycerol-saline buffer (30 mL) and passed through a 330-micron filter to create each FMT unit and stored at −80° C. For administration of FMT, three FMT units (90 mL total) were thawed and instilled rectally using a retention enema. The enema was administered in the left lateral decubitus position and retained for 30 minutes. Placebo is normal saline given and retained in the same manner.

### Microbiota Analysis

Microbial DNA was isolated from stool samples as previously described in Liag et al. Front Integr Neurosci 2018; 12:33.

### Bacterial Ribosomal RNA Gene Sequencing

The V1 and V2 variable regions of the bacterial 16S ribosomal RNA (rRNA) gene were sequenced using Multitag fusion primers and sequenced on an Ion Torrent Personal Genome Machine next-generation sequencer as described in Bajaj et al. Hepatology 2017; 66:1727-1738.

### Bioinformatics Analysis

16S rRNA gene sequence data were utilized for bioinformatics analysis. Fastq files were demultiplexed using custom PERL scripts and sequences were filtered for quality scores and length. The trimmed 16S sequences were clustered into operational taxonomic units (OTUs) using the RDP11 Bayesian Classifier bootstrap values greater than 60. The taxonomic identity of reference sequences was determined using the RDP11 Classifier online tool. George Mason University Metabiome Portal was used to organize raw data, track clinical metadata, and track analysis. The portal consists of a Drupal based interface wrapped around a MYSQL database that uses PHP to manage the relational database. The system has built in safeguards to curate the data, keep is secure, and to assure quality control. Data for this project was analyzed through these pipelines and distributed through this interface.

### Microbial Bio-statistical Analysis

Bacterial community composition was characterized using OUT counts generated as described above. OUT counts were converted to measures of relative abundance to account for variation in sequencing coverage between samples. Statistical analysis was carried out using the statistical software package R (www.r-project.org). Changes in abundance of individual taxa were analyzed using traditional univariate non-parametric statistical methods and UNIFRAC. We used LefSe (Linear discriminant analysis Effect Size) to determine the microbial taxa differences between groups (Pre vs post FMT and post-FMT vs post-placebo).

### Bacterial Taxon Analysis

A Bayesian classifier with a posterior probability was used to identify query sequence based on the occurrence of seven-length base pair subsequences in the rRNA database. The raw data was filtered using a 180 base pair cutoff. The abundances of the bacterial identifications were then normalized using a custom PERL script and taxa present at >1% of the community were tabulated. For this approach, a 1% cut off was chosen based on an a priori assumption that taxa present in <1% of the community vary between individuals and have minimal contribution to the functionality of that community and 2,000 reads per sample will only reliably identify community components that are greater than 1% in abundance. The underlying a priori assumption for this filtering is that the low abundance components of the community vary between individual subjects and will not contribute significantly to the functionality of the gut mucosal microbiome. Additionally, the Quantitative Insights into Microbial Ecology (QIIME2) package for UNIFRAC analysis and diversity analysis was utilized.

### Cognitive Testing

EncephalApp Stroop is a validated smartphone App version of the Stroop test. This involves presentation of an easier “Off” Stage in which subjects have to identify the color of the pound-signs presented on the phone and a more difficult “On” stage in which subjects must correctly identify the color of a discordant word presented. For example, the word “GREEN” will be presented in blue colored letters and the correct response would be blue and not green. The App has two practice runs and requires 5 correct runs in the Off and On Stage. The total time required for 5 correct On and 5 correct Offstage runs is the “OffTime+OnTime” which is of relevance in HE. A low OffTime+OnTime indicates better cognition. Average differences in OffTime between patients with and without cognitive impairment are >8 seconds, while that in OnTime are >7 seconds.

Psychometric hepatic encephalopathy score (PHES) is a validated five test paper-pencil battery which tests visuo-motor coordination, psychomotor speed and reaction time. It consists of the number connection test-A, number connection test B, digit symbol test, serial dotting test and a line tracing test having two components, which are time and errors. Based on population control values, the standard deviations are calculated for each sub-test and the total is added to give one value. A low score indicates better cognition.

### Results and Conclusions

Results of all testing is shown in Table 1. Alcohol craving was significantly reduced accompanied by improvement in quality of life in patients with cirrhosis with alcohol use disorder after FMT. Altering the gut-brain axis beneficially with FMT demonstrated a treatment to alleviate AUD in cirrhosis.

TABLE 1
Results of all parameters measured in Example 1.

| | Placebo Group | | FMT Group | |
|---|---|---|---|---|
| N = 10/group | Pre | Post | Pre | Post |
| MELD | 9.5 ± 2.8 | 8.3 ± 2.6 | 9.3 ± 2.6 | 8.6 ± 2.8 |
| WBC | 6.5 ± 1.3 | 7.4 ± 1.1 | 6.2 ± 1.5 | 6.7 ± 1.4 |
| ALT | 40.2 ± 16.7 | 43.8 ± 29.5 | 40.5 ± 17.1 | 58.6 ± 44.3 |
| AST | 33.5 ± 22.2 | 31.9 ± 10.3 | 34.5 ± 18.9 | 35.5 ± 10.3 |
| Alkaline phosphatase | 106.2 ± 30.8 | 104.5 ± 28.2 | 119.8 ± 21.4 | 116.0 ± 47.7 |
| Course | | | | |
| Hospitalizations | — | 2 | — | 0 |
| ACQ (high = worse) | 2.7 (1.8-4.7) | 3.0 (2.2-5.4) | 3.1 (2.4-4.5) | 2.5 (2.1-4.3)* |
| Total SIP (high = worse) | 12.4 ± 11.7 | 10.9 ± 11.1 | 12.3 ± 8.8 | 9.0 ± 10.1* |
| Physical SIP (high = worse) | 11.2 ± 9.7 | 8.6 ± 9.6 | 8.7 ± 8.0 | 8.2 ± 7.9 |
| Psychosocial SIP (high = worse) | 11.3 ± 14.4 | 10.0 ± 14.2 | 12.2 ± 13.0 | 7.0 ± 7.8* |

*p < 0.05 pre vs post

### Example 2

An altered gut-liver-brain axis underlies AUD. AUD can result in cirrhosis and often patients continue to drink despite cirrhosis development. FMT can change gut microbiota and brain function in cirrhosis, but its effect on AUD is unclear. This Example of the invention demonstrates safety of FMT and its effect on alcohol craving in cirrhotic patients with AUD who continue to drink in a clinical trial.

### Methods

Cirrhotic patients with AUD (diagnosed as having a score of AUDIT-10>8) with several unsuccessful attempts at rehabilitation, (MELD<17), without alcoholic hepatitis were randomized 1:1 into placebo or FMT in a blinded randomized trial. FMT was from one donor containing Lachnospiraceae and Ruminococcaceae. FMT/placebo were administered once via enema and pts followed for 30 days. Safety, adverse events (AE), alcohol craving questionnaire (ACQ: high=worse) and Sickness impact profile (SIP) for quality of life (QOL; has total, physical & psychosocial score, high=worse) were administered at baseline and day 30 post-intervention. Safety visits were carried out at days 1, 8, 15 and 30 post-intervention.

### Results

### Baseline

20 men with AUD Cirrhosis (65±6.4 yrs, MELD 8.9±2.7), median (IQR) AUDIT of 16.0 (12.0) underwent procedures and follow-up. Groups were evenly matched for disease severity, age (67.1±5.2 vs 62.9±7.1, p=0.15), AUDIT score (15.5±7.7 vs 16.7±7.9, p=0.74), QOL and alcohol craving.

### Course

Two patients in the placebo group were hospitalized within 30 days (1 hyponatremia & 1 atrial fibrillation); none in the FMT group. No AEs were noted. No changes in safety labs were seen.

### Patient-Reported Outcomes

A significant reduction in alcohol craving and improvement in total and psychosocial SIP was seen only in the FMT group, measured by the parameters shown in Table 2.

TABLE 2
Blood indices and psychosocial SIP.

| | Placebo (n = 10) | | FMT (n = 10) | |
|---|---|---|---|---|
| *p < 0.05 pre vs post | Pre | Post | Pre | Post |
| MELD | 9.5 ± 2.8 | 8.3 ± 2.6 | 9.3 ± 2.6 | 8.6 ± 2.8 |
| WBC | 6.5 ± 1.3 | 7.4 ± 1.1 | 6.2 ± 1.5 | 6.7 ± 1.4 |
| ALT | 40.2 ± 16.7 | 43.8 ± 29.5 | 40.5 ± 17.1 | 58.6 ± 44.3 |
| AST | 33.5 ± 22.2 | 31.9 ± 10.3 | 34.5 ± 18.9 | 35.5 ± 10.3 |
| Alkaline phosphatase | 106.2 ± 30.8 | 104.5 ± 28.2 | 119.8 ± 21.4 | 116.0 ± 47.7 |
| Course | | | | |
| Hospitalizations | — | 2 | — | 0 |
| ACQ (high = worse) | 2.7 (1.8-4.7) | 3.0 (2.2-5.4) | 3.1 (2.4-4.5) | 2.5 (2.1-4.3)* |
| Total SIP (high = worse) | 12.4 ± 11.7 | 10.9 ± 11.1 | 12.3 ± 8.8 | 9.0 ± 10.1* |
| Physical SIP (high = worse) | 11.2 ± 9.7 | 8.6 ± 9.6 | 8.7 ± 8.0 | 8.2 ± 7.9 |
| Psychosocial SIP (high = worse) | 11.3 ± 14.4 | 10.0 ± 14.2 | 12.2 ± 13.0 | 7.0 ± 7.8* |

FIG. 1 shows the scoring for alcohol craving pre- and post-treatment with FMT or placebo. Alcohol craving was significantly reduced accompanied by improvement in quality of life in patients with cirrhosis with alcohol use disorder after FMT. Altering the gut-brain axis beneficially with FMT demonstrate that can alleviate AUD in cirrhosis.

### Example 3

### Methods

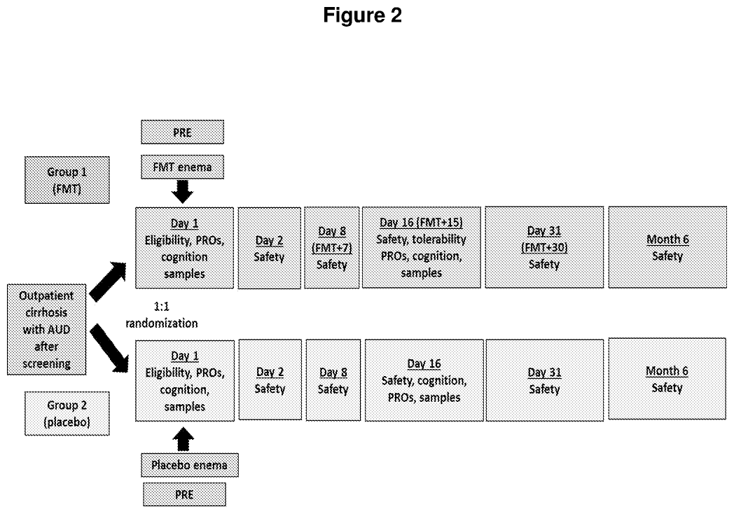

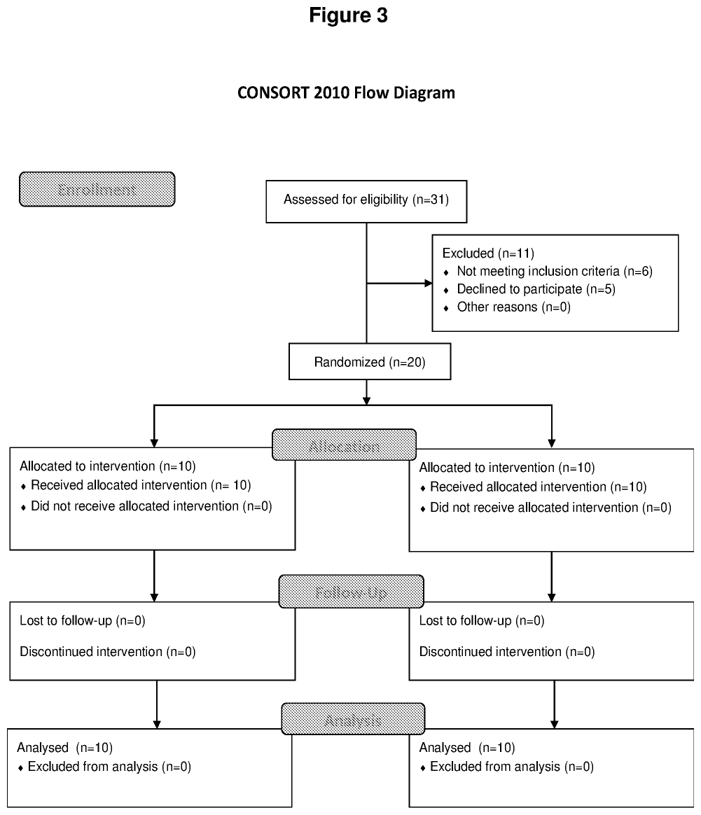

Outpatients with cirrhosis and AUD according to DSM-5 Criteria and AUDIT-10≥8 at screening were enrolled into a randomized placebo-controlled Phase 1 trial at the McGuire VA Medical Center after written informed consent. The trial was registered at www.clinicaltrials.gov as NCT03416751 and was performed under a Phase 1 FDA investigational new drug (IND). The overall schema is shown in FIG. 2 and detailed eligibility criteria are in Table 3. FIG. 3 shows a flow diagram tracking participation throughout the study.

TABLE 3
Detailed eligibility criteria for NCT03416751 FDA Phase 1 Trial.

Inclusion criteria:

| 1. | Cirrhosis diagnosed by any one of the following in a patient with chronic liver disease. | |
|---|---|---|
| | a. | Liver Biopsy. |
| | b. | Radiologic evidence of varices, cirrhosis or portal hypertension. |
| | c. | Laboratory evidence of platelet count <100,000 or AST/ALT ratio >1. |
| | d. | Endoscopic evidence of varices or portal gastropathy. |
| | e. | Transient elastography suggestive of cirrhosis. |

| | |
|---|---|
| 2. | Age between 21 and 75. |
| 3. | Able to give written, informed consent (demonstrated by mini-mental status exam >25 at the |
| | time of consenting). |
| 4. | Alcohol as a major cause of cirrhosis AND Continued sustained drinking pattern with AUDIT |
| | score >8 in the last month and fulfilling DSM-5 criteria for alcohol misuse. |
| 5. | Unable or unwilling to get mental health attention to quit alcohol (at least 3 months period of |
| | referrals to Substance abuse programs or other alcohol treatment approaches). |

Exclusion criteria:

| | |
|---|---|
| 1. | Model for End-Stage Liver Disease (MELD) score >17. |
| 2. | Child Class C. |
| 3. | WBC count <1000 cells/mm3. |
| 4. | Platelet count <50,000/mm3. |
| 5. | Unclear history of alcohol consumption. |
| 6. | TIPS in place for < one month. |
| 7. | HE episode within a month prior to the study. |
| 8. | Currently on absorbable antibiotics. |
| 9. | Infection at the time of the FMT (diagnosed by blood culture positivity, urinalysis, paracentesis |
| | as needed). |
| 10. | Hospitalization for any non-elective cause within the last 1 month. |
| 11. | Patients who are aged >75 years. |
| 12. | Patients who are pregnant or nursing (will be checked using a urine pregnancy test). |
| 13. | Patients who are incarcerated. |
| 14. | Patients who are incapable of giving their own informed consent. |
| 15. | Patients who are immuno-compromised due to the following reasons: |

| | |
|---|---|
| a. | HIV infection (any CD4 count). |
| b. | Inherited/primary immune disorders. |
| c. | Current or recent (<3 mos) treatment with anti-neoplastic agent. |
| d. | Current or recent (<3 mos) treatment with any immunosuppressant medications. |

[including but not limited to monoclonal antibodies to B and T cells, anti-TNF
agents, glucocorticoids, antimetabolites (azathioprine, 6-mercaptopurine),
calcineurin inhibitors (tacrolimus, cyclosporine), mycophenolate mofetil].
Subjects who are otherwise immunocompetent and have discontinued any
immunosuppressant medications 3 or more months prior to enrollment may be
eligible to enroll.

| | |
|---|---|
| 16. | Patients with a history of severe (anaphylactic) food allergy. |
| 17. | Patients who have previously undergone FMT. |
| 18. | Patients on renal replacement therapy. |
| 19. | Patients who are unwilling or unable to hold the enemas. |
| 20. | Patients with untreated, in-situ colorectal cancer. |
| 21. | Patients with a history of chronic intrinsic GI diseases such as inflammatory bowel disease |
| | (ulcerative colitis, Crohn's disease or microscopic colitis), eosinophilic gastroenteritis, celiac |
| | disease or irritable bowel syndrome. |
| 22. | Major gastro-intestinal or intra-abdominal surgery in the last three months. |
| 23. | Unable to comply with protocol requirements. |
| 24. | Patients who are American Society of Anesthesiologists (ASA) Physical Status classification |
| | IV and V. |
| 25. | Patients with acute illness or fever on the day of planned FMT will be excluded with the option |
| | of including that subject at a future date. |
| 26. | Any conditions for which, in opinion of MD, the treatment may pose a |
| | health risk. |
| 27. | C. difficile in the stool at baseline (qPCR). |
| 28. | Grade 2-4 or complicated hemorrhoids. |

The study was a blinded, randomized, placebo-controlled Phase 1 trial where subjects underwent screening visits to confirm eligibility after providing written informed consent. This was followed by the first visit where cognitive testing using psychometric hepatic encephalopathy score (PHES) and EncephalApp Stroop, Alcohol craving questionnaire-Short form (ACQ-SF), QOL assessment using Sickness Impact Profile (SIP), blood for safety laboratories (MELD score, blood count, hepatic function and basic metabolic panel), serum interleukin-6 (IL-6) and lipopolysaccharide-binding protein (LBP), short-chain fatty acids (SCFA) from stool and plasma, urine for ethyl-glucuronide (Etg) and creatinine (Assaygate; Ijamsville, Md.), and stool for microbiota were collected and processed according to published techniques.

The ACQ-SF has 12 brief questions on a scale from 1-7 about alcohol craving, which yields a total ACQ score. A high score indicates higher craving. The SIP was used for assessing health-related quality of life (QOL). It has a total score and psychosocial and physical domains. A high score indicates worse QOL. PHES consists of 5 sub-tests and is scored as a composite of these scores beyond healthy controls. EncephalApp Stroop has 2 components, the easier OffTime and more difficult OnTime, as described in previous Examples. Lower completion time reflects good performance on EncephalApp. Dietary recall was performed at each visit.

Randomization was performed in a 1:1 manner by the McGuire VA Medical Center Investigational Pharmacy using the www.randomizer.org program. Subjects were unaware of their allocation and the person assessing outcomes (determining safety, administering and interpreting cognitive testing and questionnaires) was blinded towards the assignment. The FMT/placebo administrators were not blinded but neither they nor the pharmacy communicated with the assessor about the assignment.

Aliquots of 90 ml (27 grams of stool) of the FMT material from the donor containing approximately 2.7e¹² colony forming units was administered via enema with the patients being on their left side in the FMT-assigned subjects. Patients in the control group received 90 ml of placebo administered in the same manner. The enema was retained for 30 minutes in each patient with the patient continuing to remain on their left side while the enema tube in the rectum stabilized by the study staff for the period. Patients did not become aware of their group assignments due to the careful positioning of the product and disposal of the equipment. Using the protocol provided by OpenBiome (OpenBiome; Cambridge, Mass.), FMT material was purchased from OpenBiome where donor selection was performed to maximize the Lachnospiraceae and Ruminococcaceae that were lacking in study participants.

Subjects were asked to complete a daily symptom diary and were interviewed pre-FMT and days 1, 7, 15 and 30 post-FMT for assessment regarding adverse events and safety. Potential adverse events were graded from 0 through 4 using an Adverse Event Grading Chart, such as the grading chart example shown in Table 4, to make any determination regarding disposition and/or follow-up, as needed.

TABLE 4
Adverse Event grading chart
Participant ID: Date: Time:
Randomization: Study Start Date: Date
*Volunteer's responses will be marked on grading sheet by circling the appropriate grade or indicating no symptoms.

| | | | | |
|---|---|---|---|---|
| Parameter | | | | Grade 4 |
| Compared to | Grade 1 | Grade 2 | Grade 3 | Potentially Life- |
| Baseline | Mild | Moderate | Severe | Threatening |

GUIDE TO ESTIMATING SEVERITY GRADE

| | | | |
|---|---|---|---|
| Symptoms with | Symptoms with | Symptoms with | Symptoms causing |
| minimal change | moderate change | severe change from | inability to perform |
| from baseline | from baseline | baseline causing | basic self-care |
| causing no or | causing greater | inability to perform | functions OR |
| minimal | than minimal | usual social & | Medical or operative |
| interference | interference with | functional activities | intervention indicated |
| with usual social | usual social & | | to prevent permanent |
| & functional | functional | | impairment, |
| activities | activities | | persistent disability, |
| | | | or death |

SYMPTOM SPECIFIC SEVERITY GRADE

| | | | | |
|---|---|---|---|---|
| Fever (oral) | (99.9-100.5° F.) | (100.6-101.5° F.) | (101.6.-104° F.) | >104° F. |
| Diarrhea | Transient or | Persistent episodes of | Bloody diarrhea | Life-threatening |
| | intermittent episodes | unformed to watery stools | OR Increase of ≥7 | consequences (e.g., |
| | of unformed stools OR | OR Increase of 4-6 stools | stools per 24-hour | hypotensive shock) |
| | Increase of ≤3 stools | over baseline per 24- | period OR IV fluid | |
| | over baseline per | hour period | replacement indicated | |
| | 24-hour period | | | |
| Nausea | Transient (<24 hours) or | Persistent nausea resulting | Persistent nausea resulting | Life-threatening |
| | intermittent nausea with | in decreased oral | in minimal oral intake | consequences (e.g., |
| | no or minimal interference | intake for 24-48 hours | for >48 hours OR Aggressive | hypotensive shock) |
| | with oral intake | | rehydration indicated (e.g., | |
| | | | IV fluids) | |
| Vomiting | Transient or intermittent | Frequent episodes of | Persistent vomiting resulting | Life-threatening |
| | vomiting with no or minimal | vomiting with no or | in orthostatic hypotension OR | consequences (e.g., |
| | interference with oral intake | mild dehydration | Aggressive rehydration | hypotensive shock) |
| | | | indicated (e.g., IV fluids) | |
| Distension, bloating, | Asymptomatic | Symptomatic, but not | Symptomatic, interfering | — |
| abdominal discomfort | | interfering with GI | with GI function | |
| | | function | | |
| Abdominal Pain | Symptoms causing no or | Symptoms causing greater | Symptoms causing inability | Disabling pain causing |
| | minimal interference | than minimal interference | to perform usual social & | inability to perform |
| | with usual social & | with usual social & | functional activities | basic self-care functions |
| | functional activities | functional activities | | OR life threatening |
| | | | | consequences (i.e. |
| | | | | acute peritonitis) |
| Dehydration | Increased oral fluids | IV fluids indicated <24 hours | IV fluids indicated >24 hours | Life-threatening consequences |
| | indicated; dry mucous | | | (e.g. hemodynamic collapse) |
| | membranes; diminished skin | | | |
| | turgor | | | |
| Constipation | NA | Persistent, requiring | Obstipation with manual | Life-threatening |
| | | regular use of dietary | evacuation indicated | consequences |
| | | modifications, laxatives, | | (e.g., obstruction) |
| | | or enemas | | |
| Procedural Enema | Rectal discomfort, | Rectal discomfort, | Gross hematochezia | Bowel perforation |
| | minor bleeding <24 hours | minor bleeding >24 hours | | |
| | on toilet paper | on toilet paper | | |
| Change in mental status | No change in orientation | Disorientation to | Disorientation to place | Coma |
| (West-Haven Criteria) | and no asterixis | time, asterixis | or person, asterixis, | |
| | | | lethargy and stupor | |
| MELD score increase | <3 | 3-7 | >8 | NA |
| (INR, bilirubin and | | | | |
| creatinine) | | | | |
| Serum WBC | NA | NA | >12,000/ml | NA |
| | | | or <1000/ml | |
| AST | NA | 5.0 to < 10.0 × ULN | ≥10.0 × ULN | NA |
| ALT | NA | 5.0 to < 10.0 × ULN | ≥10.0 × ULN | NA |
| Alkaline phosphatase | NA | 5.0 to < 10.0 × ULN | ≥10.0 × ULN | NA |

Disposition/Followup:

Safety and adverse events for placebo and FMT groups were also compiled. Data collected at pre-FMT and on day 1 post-FMT are shown in Table 5, data collected on days 7 and 15 post-FMT are shown in Table 6, and data collected on day 30 post-FMT are shown in Table 7.

TABLE 5
Changes in laboratory values and symptoms at each
safety visit, pre-FMT and day 1 post-FMT.

| | Pre-FMT | | Day 1 Post-FMT | |
|---|---|---|---|---|
| n = 10 for each group | Placebo | FMT | Placebo | FMT |

Laboratory Values

| | | | | |
|---|---|---|---|---|
| MELD | 9.5 ± 2.8 | 9.3 ± 2.6 | 9.1 ± 2.6 | 9.0 ± 3.3 |
| WBC (×10³/ml) | 6.5 ± 1.3 | 6.2 ± 1.5 | 6.0 ± 1.6 | 7.3 ± 1.6 |
| ALT (IU/L) | 40.2 ± 16.7 | 40.5 ± 17.1 | 32.1 ± 16.9 | 32.0 ± 9.7 |
| AST (IU/L) | 33.5 ± 22.2 | 34.5 ± 18.9 | 38.8 ± 16.1 | 42.5 ± 24.6 |
| Alk Phos(IU/L) | 106.2 ± 30.8 | 119.8 ± 21.4 | 114.3 ± 24.1 | 114.0 ± 32.1 |
| Albumin(g/dl) | 3.8 ± 0.4 | 3.8 ± 0.33 | 3.6 ± 0.4 | 3.7 ± 0.4 |

Symptoms (median 0-3 severity % with >1)

| | | | | |
|---|---|---|---|---|
| Fever | 0 (0%) | 0 (0%) | 0 (0%) | 0 (0%) |
| Chills | 0 (0%) | 0 (0%) | 0 (0%) | 0 (0%) |
| Diarrhea | 0 (0%) | 0 (0%) | 0 (0%) | 0 (0%) |
| Vomiting | 0 (0%) | 0 (0%) | 0 (0%) | 0 (0%) |
| Abdominal Pain | 0 (0%) | 0 (0%) | 0 (0%) | 0 (0%) |
| Constipation | 0 (0%) | 0 (0%) | 0 (0%) | 0 (0%) |
| Change in mental status | 0 (0%) | 0 (0%) | 0 (0%) | 0 (0%) |
| Total daily calories | 2321 ± 347 | 2410 ± 501 | 2238 ± 350 | 2295 ± 421 |

TABLE 6
Changes in laboratory values and symptoms at safety visits day 7 and 15 post-FMT.

| | Day 7 Post-FMT | | Day 15 Post-FMT | |
|---|---|---|---|---|
| n = 10 for each group | Placebo | FMT | Placebo | FMT |

Laboratory Values

| | | | | |
|---|---|---|---|---|
| MELD | 9.5 ± 2.7 | 8.5 ± 2.8 | 8.3 ± 2.6 | 8.6 ± 2.8 |
| WBC (×10³/ml) | 6.5 ± 3.1 | 7.3 ± 1.9 | 7.4 ± 1.1 | 6.7 ± 1.4 |
| ALT (IU/L) | 32.6 ± 20.1 | 32.2 ± 9.9 | 43.8 ± 29.5 | 58.6 ± 44.3 |
| AST (IU/L) | 37.9 ± 18.2 | 50.2 ± 37.5 | 31.9 ± 10.3 | 35.5 ± 10.3 |
| Alk Phos(IU/L) | 116.0 ± 26.3 | 111.3 ± 35.6 | 104.5 ± 28.2 | 116.0 ± 47.7 |
| Albumin(g/dl) | 3.8 ± 0.4 | 3.8 ± 0.3 | 3.7 ± 0.3 | 3.9 ± 0.4 |

Symptoms (median 0-3 severity % with >1)

| | | | | |
|---|---|---|---|---|
| Fever | 0 (0%) | 0 (0%) | 0 (0%) | 0 (0%) |
| Chills | 0 (0%) | 0 (0%) | 0 (0%) | 0 (0%) |
| Diarrhea | 0 (0%) | 0 (0%) | 0 (20%) | 0 (0%) |
| Vomiting | 0 (0%) | 0 (0%) | 0 (0%) | 0 (0%) |
| Abdominal Pain | 0 (0%) | 0 (0%) | 0 (20%) | 0 (0%) |
| Constipation | 0 (0%) | 0 (0%) | 0 (10%) | 0 (20%) |
| Change in mental status | 0 (0%) | 0 (0%) | 0 (0%) | 0 (0%) |
| Total daily calories | 2015 ± 406 | 2114 ± 519 | 2291 ± 391 | 2414 ± 527 |

TABLE 7
Changes in laboratory values and symptoms
at safety visit day 30 post-FMT.

Day 30 Post-FMT

| | | |
|---|---|---|
| n = 10 for each group | Placebo | FMT |

Laboratory Values

| | | |
|---|---|---|
| MELD | 9.5 ± 2.8 | 9.0 ± 3.4 |
| WBC (×10³/ml) | 6.5 ± 3.1 | 7.3 ± 2.0 |
| ALT (IU/L) | 34.4 ± 21.5 | 38.9 ± 15.4 |
| AST (IU/L) | 39.9 ± 15.9 | 43.0 ± 24.4 |
| Alk Phos(IU/L) | 113.5 ± 24.8 | 114.6 ± 35.1 |
| Albumin(g/dl) | 3.7 ± 0.4 | 3.8 ± 0.4 |

Symptoms (median 0-3 severity % with >1)

| | | |
|---|---|---|
| Fever | 0 (0%) | 0 (0%) |
| Chills | 0 (0%) | 0 (0%) |
| Diarrhea | 0 (0%) | 0 (0%) |
| Vomiting | 0 (0%) | 0 (0%) |
| Abdominal Pain | 0 (0%) | 0 (0%) |
| Constipation | 0 (0%) | 0 (0%) |
| Change in mental status | 0 (0%) | 0 (0%) |
| Total daily calories | 2394 ± 236 | 2417 ± 393 |

Two safety visits, on the day after intervention (day 1 post-FMT) and at one week after intervention (day 7 post-FMT), were performed. If any subject did not want to continue, their last observation was carried forward. At day 16 (15 days post-intervention), cognition, alcohol craving, stool microbiota, urinary Etg/Cr and QOL were reassessed. Serum IL-6 and LBP and stool/plasma SCFAs were also reassessed. At day 31 (30 days post-intervention or day 30 post-FMT), a safety check was performed. Finally, at 6 months a safety visit was performed again either in person, remotely or through chart review where necessitated due to COVID-19 pandemic restrictions.

Serious adverse events (SAEs) were defined as hospitalizations or emergency room visits during the study. All potential AEs and SAEs were first analyzed by the principal investigator for potential relatedness to the FMT material and forwarded to the DSMB, the IRB and FDA. The FDA mandated that all patients be followed via chart review or remotely for safety regardless of whether they were able to come in or not. Relatedness of AUD to the SAE was judged based on the medical records and presence of active alcohol drinking or withdrawal by patient interview, blood or urine alcohol levels and/or the judgement of the admitting physician. Data pertaining to alcohol abstinence were gleaned from interviews and chart review at 6 months.

The primary outcome was safety and tolerability of the FMT versus placebo with respect to SAEs. Secondary outcomes were short-term changes in microbiota composition, alcohol craving and consumption, cognition and QOL. Statistical analyses were performed using Wilcoxon signed-rank matched paired tests when comparing continuous data from baseline to post-FMT and using Mann-Whitney tests when compared post-placebo to post-FMT. To compare between groups, delta changes in SIP, alpha diversity and ACQ between FMT and placebo groups were compared using Mann-Whitney tests. Baseline analyses were compared using Mann-Whitney or unpaired t-tests as appropriate. Fisher exact tests were used to calculate proportion changes between and within groups. Microbial analyses were performed using 16srRNA sequencing of stool, changes in microbial diversity and Linear discriminant function effect size (LEfSe) was used to compare taxa pre vs post-FMT and post-FMT vs post-placebo. Correlation network analyses were performed between microbial genera, ACQ, and SCFAs pre vs post-FMT and post-FMT vs post-placebo using validated techniques in R and only correlations that were p<0.05 and r>0.7 or <0.7 were studied. Due to the Phase 1 and exploratory nature of the study, 10 subjects in each group were the target enrollment.

This study was approved by the McGuire VA Medical center IRB and all participants provided written informed consent before study procedures. Between February 2018 and October 2019, 20 men with alcohol-related cirrhosis with a mean age of 65±6.4 years, mean model for end-stage liver disease (MELD) score of 8.9±2.7, and median (IQR) AUDIT-10 (Alcohol use disorder identification test) score of 16.0(12.0) were randomized 1:1 into one-time FMT or placebo enema administration. Subjects were contacted for day 15 and 30 follow-up and were re-contacted at 6 months either in person or remotely (due to COVID-19 restrictions) as per the FDA mandate.

### Results

### Clinical Characteristics

Cirrhosis, alcohol consumption, cognitive and quality of life (QOL) variables were similar between groups at baseline, as shown in Table 8. All subjects had been referred for and undergone alcohol rehabilitation multiple times [median 2 (2-5 IQR)] unsuccessfully. Age (FMT 67.1±5.2 versus placebo 62.9±7.1 years, p=0.15) and racial distribution were comparable (7 Caucasians/3 African Americans in placebo versus 6 Caucasians/4 African Americans in FMT). All subjects were on proton pump inhibitors, were following Western non-vegetarian diet and were without recent (<2 months) or current antibiotic/probiotic use. One patient in each group was on lactulose therapy for HE; none had HE episodes in the last year and these were continued throughout the trial.

TABLE 8
Change in Clinical, Patient-reported and Cognitive Outcomes

| | Placebo | | | | FMT | | | |
|---|---|---|---|---|---|---|---|---|
| n = 10/group | Pre (baseline) | | Post (day 15) | | Pre | | Post (day 15) | |
| MELD score | 9.5 ± 2.8 | | 8.3 ± 2.6 | | 9.3 ± 2.6 | | 8.6 ± 2.8 | |
| White blood cell count (×10³/ml) | 6.5 ± 1.3 | | 7.4 ± 1.1 | | 6.2 ± 1.5 | | 6.7 ± 1.4 | |
| Aspartate aminotransferase (IU/L) | 40.2 ± 16.7 | | 43.8 ± 29.5 | | 40.5 ± 17.1 | | 58.6 ± 44.3 | |
| Alanine aminotransferase (IU/L) | 33.5 ± 22.2 | | 31.9 ± 10.3 | | 34.5 ± 18.9 | | 35.5 ± 10.3 | |
| Alkaline phosphatase (IU/L) | 106.2 ± 30.8 | | 104.5 ± 28.2 | | 119.8 ± 21.4 | | 116.0 ± 47.7 | |
| Serum albumin (g/dl) | 3.8 ± 0.4 | | 3.7 ± 0.3 | | 3.8 ± 0.33 | | 3.9 ± 0.4 | |
| Urinary Etg/creatinine (μg/g | 0.14 | (0.02-0.23) | 0.13 | (0.02-0.48) | 0.12 | (0.01-0.86) | 0.02 | (0.00-0.16) |
| creatinine Median (IQR) | | | | | | | | |

Patient-Reported Outcomes

| | | | | | | | | |
|---|---|---|---|---|---|---|---|---|
| ACQ-SF (high = worse) | 2.7 | (1.8-4.7) | 3.0 | (2.2-5.4) | 3.1 | (2.4-4.5) | 2.5 | (2.1-4.3)* |
| Median (IQR) | | | | | | | | |

| | | | | |
|---|---|---|---|---|
| Total SIP (high = worse) | 12.4 ± 11.7 | 10.9 ± 11.1 | 12.3 ± 8.8 | 9.0 ± 9.1* |
| Physical SIP (high = worse) | 11.2 ± 9.7 | 8.6 ± 9.6 | 8.7 ± 8.0 | 8.2 ± 7.9 |
| Psychosocial SIP(high = worse) | 11.3 ± 14.4 | 10.0 ± 14.2 | 12.2 ± 13.0 | 7.0 ± 7.8^{‡} |

Cognitive Testing Median (IQR)

| | | | | | | | | |
|---|---|---|---|---|---|---|---|---|
| PHES (high = better) | −6.0 | (−13.0-−3.5) | −5.5 | (−13.0-−1.75) | −5.5 | (−10.00-0.0) | −2.5 | (−9.25-1.00)^{†} |

EncephalApp (high = poor)

| | | | | | | | | |
|---|---|---|---|---|---|---|---|---|
| OffTime (seconds) | 92.1 | (86.4-104.9) | 93.7 | (78.0-104.7) | 82.9 | (76.0-97.9) | 85.3 | (75.1-99.7) |
| OnTime (seconds) | 108.3 | (98.1-126.5) | 104.3 | (89.1-149.8) | 111.5 | (91.1-124.8) | 101.7 | (93.3-110.9)^{†} |
| OffTime + OnTime (seconds) | 201.3 | (184.9-230.3) | 185.6 | (173.9-237.2) | 197.8 | (164.7-222.1) | 187.5 | (167.8-213.3) |

Data are presented as mean ± SD unless mentioned otherwise. Comparison using Wilcoxon Signed Rank Paired test within groups,
Etg: Ethylglucuronide,
ACQ-SF: alcohol craving questionnaire-short form,
SIP: sickness impact profile,
PHES: Psychometric hepatic encephalopathy score,
MELD: model for end-stage liver disease (a high score indicates poor outcome)
*p = 0.02,
^{‡}p = 0.01,
^{†}p = 0.05

### Intervention and Short-Term Follow-Up

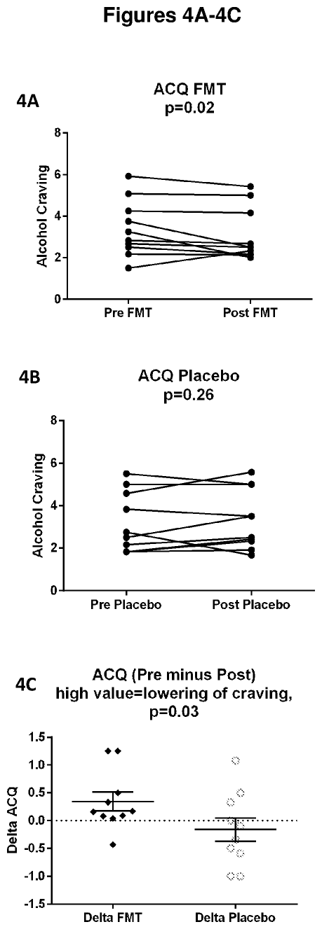

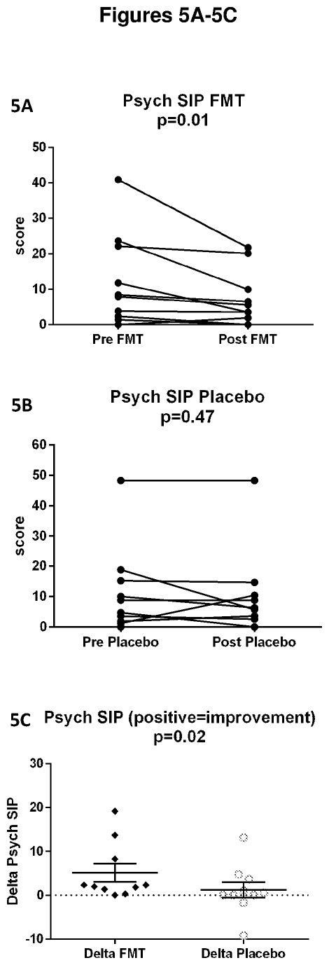

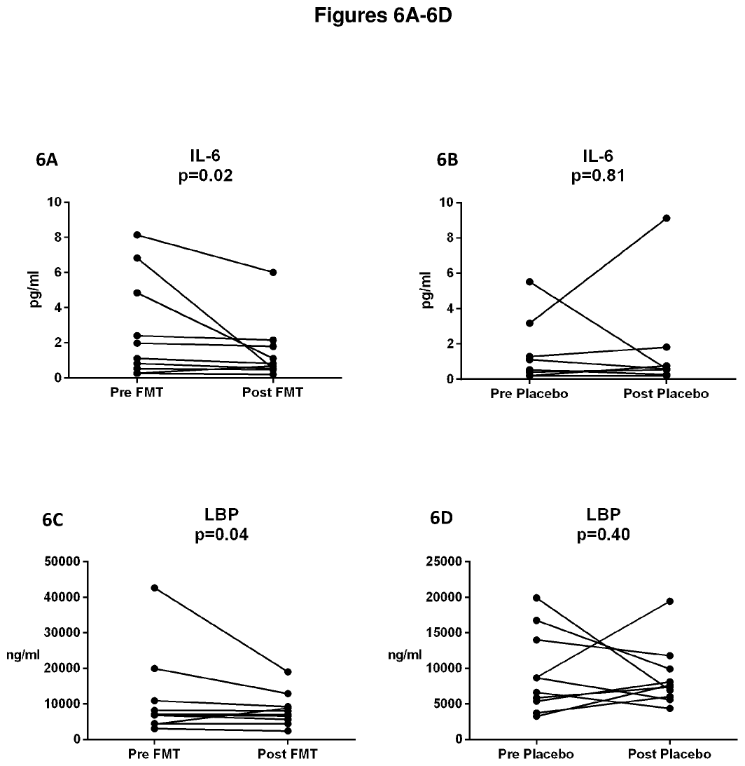

Investigational enemas were tolerated well and retained for 30 minutes. Parameters measured at intervention and short-term follow-up are shown in Table 9. No changes in dietary pattern, laboratory or symptom signals were seen over the first 30 days. A significant improvement in ACQ-SF was seen at day 15 compared to baseline in FMT-assigned participants, but not in placebo, as shown in FIGS. 4A-4C. Nine subjects in FMT reduced their craving compared to only 3 in placebo (p=0.02) and delta change was also higher in FMT. Similarly, a significant reduction in urinary Etg/creatinine in the FMT but not placebo was observed. A SIP assessment showed that the QOL improved in the FMT group only in the psychosocial and not physical domain, and change was greater than placebo-assigned patients, as shown in FIGS. 5A-5C. There was also a reduction in serum IL-6 and LBP concentration in FMT compared to baseline, shown in FIGS. 6A and 6B, respectively. Cognitively, PHES and EncephalApp OffTime+OnTime improved in FMT patients. This was primarily due to lower OnTime.

TABLE 9
Parameters for Intervention and short-term follow-up

| | Placebo (n = 10) | | | | FMT (n = 10) | | | |
|---|---|---|---|---|---|---|---|---|
| | Pre (baseline) | | Post (day 15) | | Pre | | Post (day 15) | |
| MELD score | 9.5 ± 2.8 | | 8.3 ± 2.6 | | 9.3 ± 2.6 | | 8.6 ± 2.8 | |
| White blood cell count ×10³/ml | 6.5 ± 1.3 | | 7.4 ± 1.1 | | 6.2 ± 1.5 | | 6.7 ± 1.4 | |
| Aspartate aminotransferase IU/L | 40.2 ± 16.7 | | 43.8 ± 29.5 | | 40.5 ± 17.1 | | 58.6 ± 44.3 | |
| Alanine aminotransferase IU/L | 33.5 ± 22.2 | | 31.9 ± 10.3 | | 34.5 ± 18.9 | | 35.5 ± 10.3 | |
| Alkaline phosphatase IU/L | 106.2 ± 30.8 | | 104.5 ± 28.2 | | 119.8 ± 21.4 | | 116.0 ± 47.7 | |
| Serum albumin g/dl | 3.8 ± 0.4 | | 3.7 ± 0.3 | | 3.8 ± 0.33 | | 3.9 ± 0.4 | |
| Urinary Etg/creatinine μg/g | 0.14 | (0.02-0.23) | 0.13 | (0.02-0.48) | 0.12 | (0.01-0.86) | 0.02 | (0.00-0.16) |
| creatinine Median (IQR) | | | | | | | | |

Patient-Reported Outcomes

| | | | | | | | | |
|---|---|---|---|---|---|---|---|---|
| ACQ-SF (high = worse) | 2.7 | (1.8-4.7) | 3.0 | (2.2-5.4) | 3.1 | (2.4-4.5) | 2.5 | (2.1-4.3)* |
| Median (IQR) | | | | | | | | |

| | | | | |
|---|---|---|---|---|
| Total SIP (high = worse) | 12.4 ± 11.7 | 10.9 ± 11.1 | 12.3 ± 8.8 | 9.0 ± 9.1* |
| Physical SIP (high = worse) | 11.2 ± 9.7 | 8.6 ± 9.6 | 8.7 ± 8.0 | 8.2 ± 7.9 |
| Psychosocial SIP (high = worse) | 11.3 ± 14.4 | 10.0 ± 14.2 | 12.2 ± 13.0 | 7.0 ± 7.8^{‡} |

Cognitive Testing Median (IQR)

| | | | | | | | | |
|---|---|---|---|---|---|---|---|---|
| PHES (high = better) | −6.0 | (−13.0-−3.5) | −5.5 | (−13.0-−1.75) | −5.5 | (−10.00-0.0) | −2.5 | (−9.25-1.00)^{†} |

EncephalApp (high = poor)

| | | | | | | | | |
|---|---|---|---|---|---|---|---|---|
| OffTime seconds | 92.1 | (86.4-104.9) | 93.7 | (78.0-104.7) | 82.9 | (76.0-97.9) | 85.3 | (75.1-99.7) |
| OnTime seconds | 108.3 | (98.1-126.5) | 104.3 | (89.1-149.8) | 111.5 | (91.1-124.8) | 101.7 | (93.3-110.9)^{†} |
| OffTime + OnTime seconds | 201.3 | (184.9-230.3) | 185.6 | (173.9-237.2) | 197.8 | (164.7-222.1) | 187.5 | (167.8-213.3) |

### Microbiota Changes

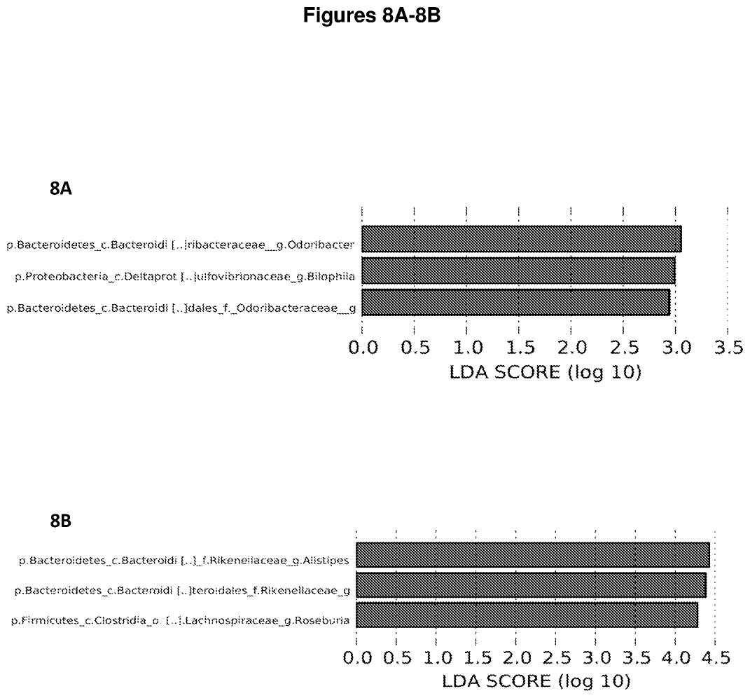

Shannon-diversity increased in the post-FMT group compared to baseline and placebo-end, shown in FIGS. 8A and 8B. On LEfSe, there was a higher relative abundance of Odoribacter and Bilophila compared to baseline in FMT, shown in FIG. 8A. At study-end, Alistipes and Roseburia were higher in FMT compared to placebo, as shown in FIG. 8B. No changes were seen between pre vs post-placebo microbiota. The post-FMT group showed increases in Lachnospiraceae and Ruminococcaceae families such as, Odoribacter, Blautia, Roseburia, in addition to other taxa such as Alistipes, Eubacterium, Lactonifactor, Oscillibacter, Anaerostipes and Bilophila. No changes within the placebo group were seen.

### Short Chain Fatty Acid Measurements

A higher plasma butyrate, isobutyrate and isovalerate with higher stool isobutyrate and trend towards higher butytate, valerate, isovalerate and hexanoate was measured in stool samples from FMT recipients compared to baseline on one-tailed testing, as shown in Table 10. In contrast, there was a reduction in stool isovalerate, and 2-methylbutyrate in stool samples from the placebo group. None of the other moieties were significant.

TABLE 10
Measurements of short-chain fatty acids in stool samples

| | Placebo (n = 10) | | FMT (n = 10) | |
|---|---|---|---|---|
| Median (IQR) | Pre (baseline) | Post (day 15) | Pre (baseline) | Post (day 15) |

Plasma (ng/ml)

| | | | | | | | | |
|---|---|---|---|---|---|---|---|---|
| Butyric acid | 136.5 | (100.9-251.0) | 138.5 | (95.9-215.8) | 118.0 | (69.5-195.5) | 167.0 | (95.1-297.0)* |
| Isobutyric acid | 638.0 | (422.5-937.3) | 689.5 | (494.8-866.3)) | 534.0 | (426.5-848.8) | 613.5 | (523.3-835.5)* |
| Acetic acid | 5595 | (2503-17575) | 7560 | (3198-41650) | 11705 | (1840-49475) | 22450 | (4240-44725) |
| Propionic acid | 449.5 | (362.3-594.5) | 461.0 | (369.3-551.8) | 427.0 | (266.5-520.5) | 386.0 | (327.5-572.5) |
| Valeric acid | 98.6 | (63.1-159.8) | 92.6 | (55.0-147.3) | 58.9 | (46.0-84.1) | 70.8 | (50.4-84.9) |
| Isovaleric acid | 93.8 | (54.1-169.5) | 82.4 | (43.1-111.8) | 81.7 | (48.3-99.2) | 101.9 | (85.8-122.3)* |
| 2-methybutyric acid | 218.5 | (109.3-309.3) | 174.0 | (138.8-252.3) | 190.0 | (153.0-248.3) | 213.0 | (165.8-273.3) |
| Hexanoic acid | 154.0 | (111.6-176.5) | 180.0 | (136.7-216.8) | 148.5 | (119.0-187.3) | 154.0 | (111.6-176.5) |

Stool (μg/g)

| | | | | | | | | |
|---|---|---|---|---|---|---|---|---|
| Butyric acid | 1070 | (477.8-2103) | 727.0 | (533.8-1528) | 718.5 | (303.1-1658) | 1140 | (453.3-2078) |
| Isobutyric acid | 233.5 | (150.8-306.3) | 235.5 | (163.8-292.0) | 135 | (100.2-193.0) | 204.0 | (120.6-273.3)* |
| Acetic acid | 3675 | (2465-4863) | 3120 | (2383-4828) | 4030 | (1365-5335) | 3375 | (1728-5565) |
| Propionic acid | 1073 | (541.8-1818) | 1180 | (822-1853) | 1535 | (562-1800) | 1630 | (915.5-1908) |
| Valeric acid | 231.5 | (103.4-407.8) | 303.5 | (189.0-493.3) | 277 | (91.2-360.3) | 370.5 | (136.3-653.5) |
| Isovaleric acid | 238.0 | (146.0-254.3) | 136.0 | (89.2-176.3)* | 128.0 | (71.4-150.8) | 169.5 | (121.9-289.5) |
| 2-methybutyric acid | 138.0 | (99.0-179.5) | 98.1 | (45.4-121.8)* | 104.0 | (50.5-135.0) | 122.0 | (87.2-204.5) |
| Hexanoic acid | 167.1 | (28.7-523.5) | 157.0 | (18.9-310.8) | 21.6 | (5.6-184.9) | 69.9 | (3.0-422.5) |

Data are presented as mean ± SD unless mentioned otherwise. Comparison using Wilcoxon Signed Rank Paired test one-tailed test within groups,
*p = 0.05

### Correlation Network Differences

The following results show changes in correlations of microbes and ACQ values in two comparisons:
1. Within FMT group: Comparing correlations before FMT to the same people after FMT
2. Between FMT and placebo group: Comparing correlations after FMT in the group that received FMT to correlations after placebo in the group that received placebo.

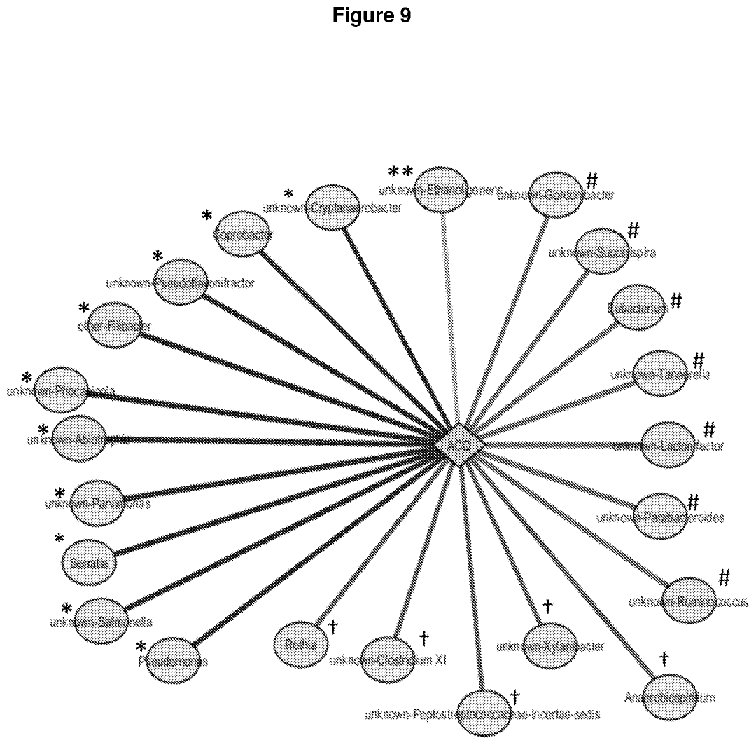

FIG. 9 shows the Post-placebo vs Post-FMT Correlation network differences centered around ACQ. A high score on ACQ indicated higher craving. Potentially beneficial genera such as Eubacterium, Lactonifactor and Ruminococcus were associated with low ACQ after FMT but not after placebo. The reverse pattern was seen with potential pathobionts such as Salmonella, Serratia and Pseudomonas. Ethanoligenens, which is associated with endogenous alcohol production, was negatively linked with ACQ in post-FMT but positive in post-placebo patients.

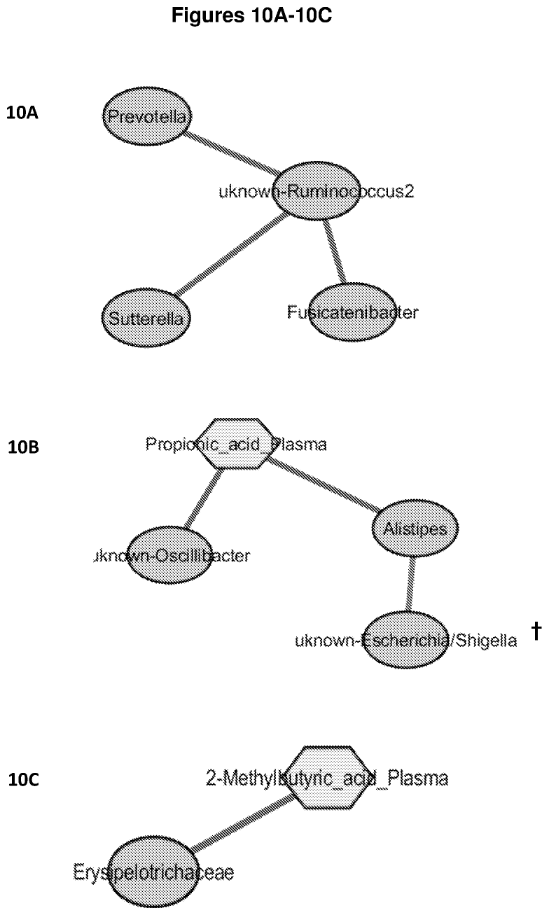

FIGS. 10A-10C show the post-placebo vs. post-FMT correlation network differences centered around SCFA. Positive linkages were found between Ruminococcaceae and Lachnospiraceae constituents and SCFA in plasma and stool post-FMT but not post-placebo. Ethanoligenens was negatively linked with hexanoate post-FMT but not placebo.

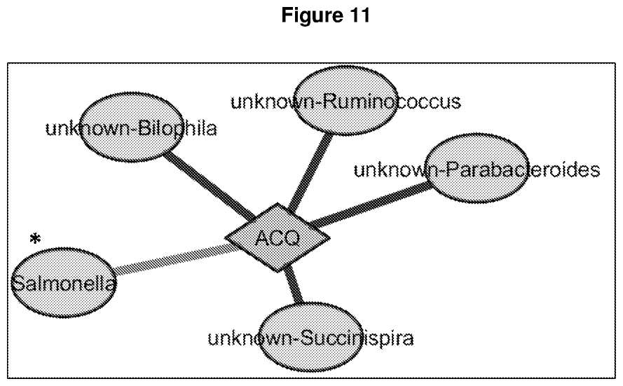

FIG. 11 shows the post-FMT correlation network differences centered around ACQ. Potentially beneficial genera and those higher in post-FMT such as Bilophila, and Ruminococcus were associated with low ACQ after FMT but not at baseline. The reverse or negative pattern was seen with potential pathobionts such as Salmonella.

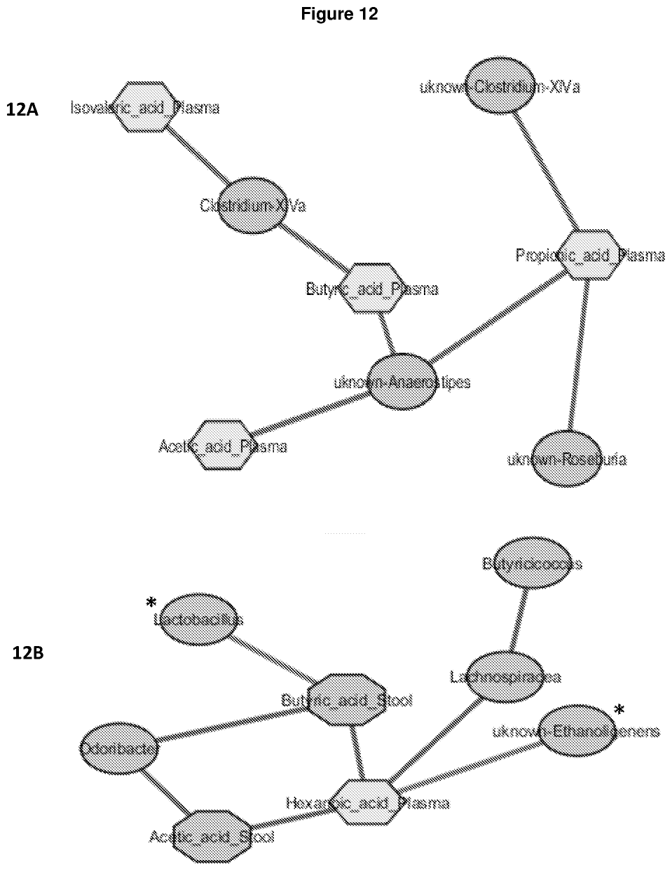

FIGS. 12A and 12B show pre-FMT vs post-FMT correlation network differences centered around SCFA. Those positively linked included the beneficial microbiota species Oscillibacter and Alistipes, which were higher post-FMT with plasma propionate, shown in FIG. 12A. The pathobiont species Erysipelothricaceae was associated with 2-methylbutyric acid post-FMT, shown in FIG. 12B.

Thus, post-FMT ACQ-SF was negatively associated with Ruminococcaceae genera compared to pre-FMT or post-placebo, and also with Proteobacteria genera. The reverse (negative) pattern was seen with potential pathobionts such as Enterococcus and Pseudomonas. Ethanoligenens, which is associated with endogenous alcohol production, was negatively linked with ACQ in post-FMT but positive in post-placebo patients. Beneficial genera and those higher in post-FMT such as Bilophila, and Ruminococcus were associated with low ACQ after FMT but at baseline. The reverse (negative) pattern was seen with potential pathobionts such as Salmonella.

Furthermore, when SCFA moieties were analyzed post-placebo compared post-FMT, plasma and stool SCFA, including butyrate, were positively linked with genera belonging to beneficial species belonging to Lachnospiraceae and Ruminococcaceae families in post-FMT. In addition, other beneficial genera that were higher post-FMT such as Odoribacter were also associated with SCFA and low ACQ. Pathobionts Ethanoligenens in contrast was negatively linked with an increase in hexanoic acid and Streptococcus was negatively associated with Blautia and Lactobacillus was negatively linked with an increase in butyrate. Comparing pre vs post-FMT, there was a greater positive linkage between Lachnospiraceae and Ruminococcaceae constituents post-FMT as well as a linkage with Alistipes and propionic acid. Also, Bilophila, which was higher post-FMT was associated with lower ACQ.

### Long-Term Follow-Up

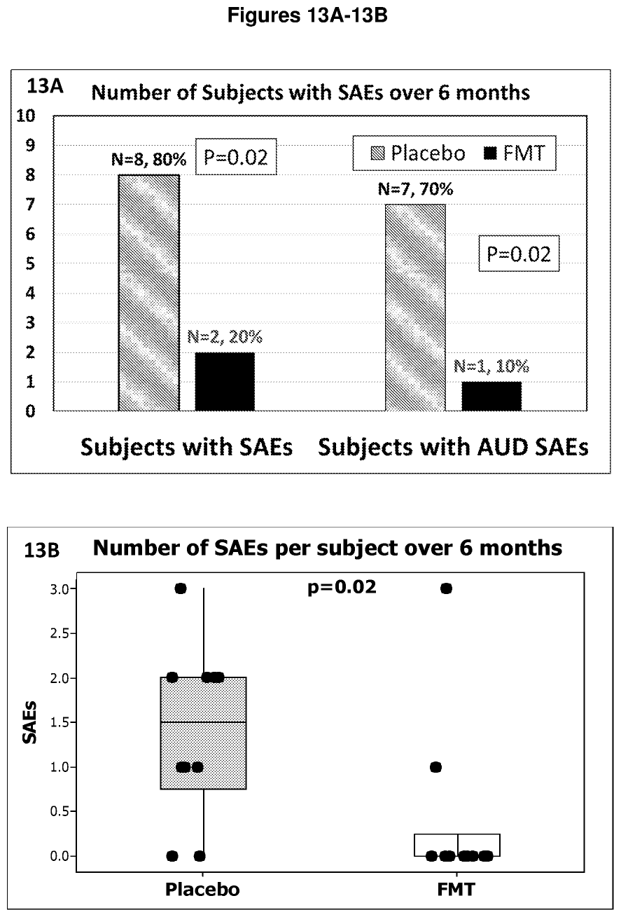

Over the long-term, eight placebo and two FMT-assigned patients developed serious adverse events (SAE), as shown in FIG. 14A (p=0.02). The median (IQR) SAE per patient were 0 (0.25) in FMT and 1.5 (1.25) in placebo (p=0.02, FIG. 13B). The average time to events was 77±53 days post-intervention. The SAE in the two FMT-assigned patients were considered unrelated to FMT transfer by the Data Safety Monitoring Board. In the placebo group, 7 patients developed ≥1 AUD-related event, as shown in Table 11 and FIG. 13B.

TABLE 9
Details of Serious Adverse Events (SAE) between groups.

| | | | | | | |
|---|---|---|---|---|---|---|
| | | | | | | Continued |
| | | | | | Alcohol- | problem |
| Subject | | | | Days after | related | drinking at 6 |
| Number | Assignment | SAEs? | Details of SAEs | Intervention | SAE? | months? |

FMT-assigned group

| | | | | | | |
|---|---|---|---|---|---|---|
| 1 | FMT | No | None | NA | NA | Yes |
| 5 | FMT | Yes | Exacerbation of | 40 | No | Yes |
| | | | heart failure due | | | |
| | | | to diuretic non- | | | |
| | | | adherence | | | |
| 6 | FMT | No | No | NA | NA | Yes |
| 7 | FMT | No | No | NA | NA | Yes |
| 10 | FMT | No | No | NA | NA | No |
| 13 | FMT | No | No | NA | NA | Yes |
| 14 | FMT | Yes | Pericarditis | 45 | No | Yes |
| | | | Post- | 154 | No | |
| | | | polypectomy | 179 | Yes | |
| | | | colonic bleed | | | |
| | | | Alcohol relapse | | | |
| 15 | FMT | No | No | NA | NA | Yes |
| 16 | FMT | No | No | NA | NA | No |
| 17 | FMT* | No | No | NA | NA | No |

Placebo group

| | | | | | | |
|---|---|---|---|---|---|---|
| 2 | Placebo | Yes | Right foot pain | 70 | No | Yes |
| | | | and injury | 105 | Yes | |
| | | | Fall hitting head | | | |
| 3 | Placebo | Yes | Right knee | 112 | No | Yes |
| | | | swelling and | 119 | Yes | |
| | | | injury | | | |
| | | | Altered mental | | | |
| | | | status | | | |
| 4 | Placebo | Yes | Cellulitis | 106 | No | Yes |
| 8 | Placebo | Yes | Atrial | 44 | Yes | Yes |
| | | | fibrillation | 162 | Yes | |
| | | | Alcohol | | | |
| | | | withdrawal | | | |
| 9 | Placebo | No | No | NA | NA | Yes |
| 11 | Placebo | No | No | NA | NA | Yes |
| 12 | Placebo | Yes | Vehicle accident | 152 | Yes | No |
| 18 | Placebo | Yes | Atrial | 25 | Yes | Yes |
| | | | fibrillation | | | |
| 19 | Placebo | Yes | Fall and hip | 164 | Yes | Yes |
| | | | fracture | | | |
| 20 | Placebo* | Yes | Hyponatremia | 14 | Yes | Yes |
| | | | Alcohol relapse | 44 | Yes | |
| | | | Alcohol relapse | 90 | Yes | |
| | | | Alcohol relapse | 96 | Yes | |
| | | | Sepsis | 155 | No | |

NA: not applicable,
FMT: fecal microbiota transplant
*patients on HE therapy

Patients with any AUD-related SAE were significantly lower in the FMT versus placebo group (1 vs 7, p=0.02). The time to first AUD-related events was 89±62 days in placebo while the only AUD-related event in FMT group occurred at day 179 post-intervention. AUD-related events continued throughout the 6-month period, indicating continued alcohol misuse in most placebo-assigned patients. At 6 months, three patients in the FMT and one in placebo group had stopped drinking based on interview and chart review. Through chart review and patient contact, the patients who continued to misuse alcohol had a steady rate of drinking without binges. One patient with prior HE was randomized to placebo and one to FMT. The FMT-assigned patient did not get hospitalized, while the patient with HE assigned to placebo was admitted for alcohol-related but not HE-related reasons multiple times. Both patients continued their therapy throughout the trial and no changes in their mentation were observed.

Changes in the gut-brain axis are found in several behavioral and addictive disorders, but translation into clinical practice by microbial modulation was previously unclear. Example 2 included AUD patients with cirrhosis who had failed, and were unwilling to participate in usual therapies, which left adequate equipoise in studying FMT associated with potential risks in this population. The use of FMT to affect brain functional change builds from prior trials in patients with cirrhosis and HE.

The part of the EncephalApp which interrogates cognitive flexibility and inhibitory control, i.e., OnTime, improved post-FMT, rather than the more basic OffTime part of the App, which relies mostly on psychomotor speed. Inhibitory control is often impaired in addictive disorders and this focused benefit may associate with favorable craving changes. Moreover, this was accompanied by improved psycho-social but not physical QOL. The improvement centered around higher mental functions and psychosocial QOL demonstrates that the impact of FMT may be selective and not an overall improvement in QOL. ACQ-SF captures the stated intention to use alcohol, which largely tracks urges and desires to drink, while urinary Etg/creatinine determines actual intake. These findings demonstrate that both perceived and actual short-term alcohol craving and use may be reduced by FMT.

The microbial changes post-FMT support the short-term findings. Increased alpha-diversity was accompanied by increased relative abundance of butyrate-producing genera such as Roseburia and Odoribacter post-FMT. This is important because Alistipes and Odoribacter are lower in alcohol-related cirrhosis, while butyrate producers are lower in AUD with and without cirrhosis. Alistipes and Bilophila also decrease with advancing liver disease and grow with higher fat availability, which is associated with protection from alcohol-induced intestinal and liver injury. Moreover, craving amelioration was also associated with Ruminococcaceae genera post-FMT. These data extend prior experience that demonstrates increase in beneficial taxa after alcohol abstinence into the realm of FMT using a donor enriched in taxa that are deficient in this population.

The Examples of the invention demonstrate a trend towards higher stool and plasma SCFA in the FMT group that were linked positively with Lachnospiraceae and most Ruminococcaceae constituents. The higher trend towards butyrate and isobutyrate and their association with Lachnospiraceaeae and Ruminococcaceae post-FMT points towards the Firmicutes that were higher in the donor material. Several taxa such as Alistipes and Odoribacter that increased post-FMT were also positively linked with SCFA levels. Other SCFA-producing taxa such as Eubacterium and Anaerostipes were also associated with lower ACQ and with SCFAs post-FMT(32). Ethanoligenens, an ethanol-producing genus of Ruminococcaceae, in contrast was negatively linked with SCFAs and positively with ACQ showing that these linkages were selective away from alcohol-producing and towards SCFA-producing taxa. Likely due to small numbers and inter-individual variations, the effects on SCFA levels themselves were not striking but their linkages with bacteria that increased post-FMT could indicate these moieties as potential mechanisms behind gut-brain axis improvement. Prior studies correlate with the findings in the Examples of the invention, showing that SCFA modulation engages the gut-brain axis in animal models and humans with addiction disorders.

Moreover, these changes were accompanied by a short-term reduction in systemic inflammation, as indicated by IL-6, and potential reduction in intestinal permeability with lower LBP in the FMT recipients but not the placebo group. Without being bound to theory as to the exact gut-brain mechanism of these short-term benefits in craving and brain function, SCFAs, reduction in systemic inflammation and relatively strengthened intestinal barrier are contributory. These findings ultimately demonstrate that the short-term impact of FMT in AUD patients extends beyond the gut lumen with bioactive metabolite and intestinal barrier changes. Given the short-term changes in microbial function, intestinal barrier and inflammation, administration of multiple FMTs may used to achieve a long-term effect.

In patients with both AUD and cirrhosis, who are prone to events focused on either condition, most events in placebo-assigned patients were focused on AUD and not cirrhosis. This highlights the effect of the intervention against AUD and the severity of the underlying AUD in this population. Despite one patient in each group being on HE therapy, there were no changes pertaining to HE during the follow-up for any subject. While there was a trend towards greater abstinence in the FMT-as signed group at 6 months, the mechanism of this longer-term change is unclear. The lower event rate could reflect the reduction but not the absence of alcohol intake in more patients in the FMT versus placebo group. These findings are in line with prior long-term studies of FMT in hepatic encephalopathy and alcoholic hepatitis and extend these into patients with AUD.

The Examples herein are limited by the phase 1 nature which is not powered for efficacy and the resultant small sample size. In addition, the short-term microbial and alcohol consumption assessment without a daily record of alcohol use over the long-term is a limitation; thus, clinical follow-up, chart review and admissions pertaining to AUD were relied upon. Patients in these Examples were men and further studies in women, who have a different AUD susceptibility pattern are needed. Given the favorable short-term microbial changes and relatively lower AUD-related outcomes in this group of patients who had continued alcohol misuse despite reaching end-organ disease, treatments of AUD without cirrhosis may also be safe and potentially provide benefit.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A method of reducing alcohol craving in a subject in need thereof, comprising the steps of
assaying a stool sample collected from the subject to identify microbiota species resident in the subject's colon and identifying the subject as being deficient in beneficial microbiota species,
obtaining a sample of fecal material from a suitable donor wherein the sample of fecal material comprises beneficial microbiota, wherein beneficial microbiota are those associated with a reduction in alcohol craving and/or consumption,
processing the sample of fecal material to produce at least one dose for fecal material transfer (FMT), and
administering the at least one dose of FMT to the subject.

2. The method of claim 1, wherein the administering step is performed as an enema comprising the at least one dose of FMT.

3. The method of claim 2, wherein the enema is retained in the colon for at least thirty minutes.

4. The method of claim 1, wherein the microbiota in the sample of fecal material comprises one or more species selected from the group consisting of Lachnospiraceae, Ruminococcaceae, Odoribacter, Blautia, Alistipes and Bilophla.

5. The method of claim 1, wherein the step of processing comprises
screening for pathogens selected from the group consisting of Clostridium difficile toxin B qualitative RT-PCR, Cyclospora and Isospora examination, ova and parasites exam with Giardia antigen EIA, Salmonella-Shigella-Campylobacter culture, Shiga toxins EIA with reflex to E. coli O157 culture and Vibrio culture, Cryptosporidium antigen EIA, Helicobacter pylori antigen EIA, stool norovirus EIA, stool rotavirus antigen detection, adenovirus antigen detection, gastroenteritis EIA, vancomycin-resistant Enterococcus culture and Microsporidia exam;
filtering the sample of fecal material;
suspending the filtered fecal material in a pharmaceutically acceptable buffered solution to produce the at least one dose of FMT.

6. The method of claim 5 further comprising the steps of
storing the at least one dose of FMT at −80° C. or below until time of administration, and
thawing the at least one dose of FMT for the administering step.

7. The method of claim 1, further comprising the steps of
obtaining a second stool sample from the subject at least 15 days after the administering step,
culturing the second stool sample to identify microbiota species resident in the subject's colon after receiving the at least one dose of FMT, and
determining whether the microbiota of the subject has been altered by receiving the at least one dose of FMT,
wherein the microbiota is beneficially altered when any of Lachnospiraceae, Ruminococcaceae, Odoribacter, Blautia, Alistipes, Roseburia, Eubacterium, Lactonifactor, Oscillibacter, Anaerostipes and/or Bilophila species are increased in the second stool sample, and any of Salmonella, Serratia, Pseudomonas and/or Ethanoligenens species are decreased in the second stool sample, or
wherein the microbiota is not beneficially altered when any of Lachnospiraceae, Ruminococcaceae, Odoribacter, Blautia, Alistipes, Roseburia, Eubacterium, Lactonifactor, Oscillibacter, Anaerostipes and/or Bilophila species are the same or not increased in the second stool sample, and any of Salmonella, Serratia, Pseudomonas and/or Ethanoligenens species are the same or increased in the second stool sample.

8. The method of claim 1, further comprising administering another dose of FMT at least once at least fifteen days after the administering at least one dose step.

9. A method of changing short-chain fatty acids (SCFA) in the colon of a subject in need thereof, comprising the steps of
assaying a stool sample from the subject to identify microbiota species resident in the subject's colon and identifying the subject as being deficient in microbiota known to increase levels of short-chain fatty acids associated with a reduction in alcohol craving and/or consumption,
obtaining a sample of fecal material from a suitable donor wherein the sample of fecal material comprises microbiota known to increase levels of short-chain fatty acids associated with a reduction in alcohol craving and/or consumption,
processing the sample of fecal material to produce at least one dose for fecal material transfer (FMT), and
administering the at least one dose of FMT to the subject.

10. The method of claim 9, wherein the administering step is performed as an enema comprising the at least one dose of FMT.

11. The method of claim 10, wherein the enema is retained in the colon for at least thirty minutes.

12. The method of claim 9, wherein the microbiota in the at least one dose of fecal material comprises one or more species selected from the group consisting of Lachnospiraceae, Ruminococcaceae, Odoribacter, Blautia, Alistipes and Bilophla.

13. The method of claim 9, further comprising the steps of
obtaining a second stool sample from the subject at least 15 days after the administering step,
culturing the second stool sample to identify microbiota species resident in the subject's colon after receiving the at least one dose of FMT, and
determining whether the microbiota species of the subject has been altered by receiving the at least one dose of FMT,
wherein the microbiota is beneficially altered when any of Lachnospiraceae, Ruminococcaceae, Odoribacter, Blautia, Alistipes, Roseburia, Eubacterium, Lactonifactor, Oscillibacter, Anaerostipes and/or Bilophila species are increased in the second stool sample, and any of Salmonella, Serratia, Pseudomonas and/or Ethanoligenens species are decreased in the second stool sample, or
wherein the microbiota is not beneficially altered when any of Lachnospiraceae, Ruminococcaceae, Odoribacter, Blautia, Alistipes, Roseburia, Eubacterium, Lactonifactor, Oscillibacter, Anaerostipes and/or Bilophila species are the same or not increased in the second stool sample, and any of Salmonella, Serratia, Pseudomonas and/or Ethanoligenens species are the same or increased in the second stool sample.

14. The method of claim 9, further comprising administering another dose of FMT at least once at least fifteen days after the administering at least one dose step.

15. The method of claim 9, wherein the step of processing comprises
screening the sample of fecal material for pathogens,
filtering the sample of fecal material, and
suspending the filtered fecal material in a pharmaceutically acceptable buffered solution to produce the at least one dose of FMT.

16. The method of claim 15, wherein the pathogens are selected from the group consisting of Clostridium difficile toxin B qualitative RT-PCR, Cyclospora and Isospora examination, ova and parasites exam with Giardia antigen EIA, Salmonella-Shigella-Campylobacter culture, Shiga toxins EIA with reflex to E. coli O157 culture and Vibrio culture, Cryptosporidium antigen EIA, Helicobacter pylori antigen EIA, stool norovirus EIA, stool rotavirus antigen detection, adenovirus antigen detection, gastroenteritis EIA, vancomycin-resistant Enterococcus culture and Microsporidia exam.

17. The method of claim 14, further comprising
storing the at least one dose of FMT at −80° C. or below until time of administration, and
thawing the at least one dose of FMT for the administering step.
